SEQUENCE LISTING (105 sequences)

SEQ ID NO: 1 - protein, 9 aa, Artificial sequence
  Synthetic polypeptide
PFPQPELPY

SEQ ID NO: 2 - protein, 9 aa, Artificial sequence
  Synthetic polypeptide
PQPELPYPQ

SEQ ID NO: 3 - protein, 9 aa, Artificial sequence
  Synthetic polypeptide
PFPQPEQPF

SEQ ID NO: 4 - protein, 9 aa, Artificial sequence
  Synthetic polypeptide
PQPEQPFPW

SEQ ID NO: 5 - protein, 9 aa, Artificial sequence
  Synthetic polypeptide
PIPEQPQPY

SEQ ID NO: 6 - protein, 15 aa, Artificial sequence
  Synthetic polypeptide
LQPFPQPELPYPQPQ

SEQ ID NO: 7 - protein, 14 aa, Artificial sequence
  Synthetic polypeptide
QPFPQPEQPFPWQP

SEQ ID NO: 8 - protein, 15 aa, Artificial sequence
  Synthetic polypeptide
PEQPIPEQPQPYPQQ

SEQ ID NO: 9 - protein, 16 aa, Artificial sequence
  Synthetic polypeptide
ELQPFPQPELPYPQPQ

SEQ ID NO: 10 - protein, 15 aa, Artificial sequence
  Synthetic polypeptide
EQPFPQPEQPFPWQP

SEQ ID NO: 11 - protein, 16 aa, Artificial sequence
  Synthetic polypeptide
EPEQPIPEQPQPYPQQ

SEQ ID NO: 12 - protein, 4 aa, Artificial sequence
  Synthetic polypeptide
PELP

SEQ ID NO: 13 - protein, 5 aa, Artificial sequence
  Synthetic polypeptide
PELPY

SEQ ID NO: 14 - protein, 9 aa, Artificial sequence
  Synthetic polypeptide
PYPQPELPY

SEQ ID NO: 15 - protein, 9 aa, Artificial sequence
  Synthetic polypeptide
PFPQPQLPY

SEQ ID NO: 16 - protein, 4 aa, Artificial sequence
  Synthetic polypeptide
PQLP

SEQ ID NO: 17 - protein, 5 aa, Artificial sequence
  Synthetic polypeptide
PQLPY

SEQ ID NO: 18 - protein, 9 aa, Artificial sequence
  Synthetic polypeptide
PYPQPQLPY

SEQ ID NO: 19 - protein, 9 aa, Artificial sequence
  Synthetic polypeptide
PFPQPQLPY

SEQ ID NO: 20 - protein, 9 aa, Artificial sequence
  Synthetic polypeptide
PQPQLPYPQ

SEQ ID NO: 21 - protein, 9 aa, Artificial sequence
  Synthetic polypeptide
PFPQPQQPF

SEQ ID NO: 22 - protein, 9 aa, Artificial sequence
  Synthetic polypeptide
PQPQQPFPW

SEQ ID NO: 23 - protein, 9 aa, Artificial sequence
  Synthetic polypeptide
PIPQQPQPY

SEQ ID NO: 24 - protein, 15 aa, Artificial sequence
  Synthetic polypeptide
LQPFPQPQLPYPQPQ

SEQ ID NO: 25 - protein, 14 aa, Artificial sequence
  Synthetic polypeptide
QPFPQPQQPFPWQP

SEQ ID NO: 26 - protein, 15 aa, Artificial sequence
  Synthetic polypeptide
PEQPIPQQPQPYPQQ

SEQ ID NO: 27 - protein, 9 aa, Artificial sequence
  Synthetic polypeptide
PFPQPDLPY

SEQ ID NO: 28 - protein, 9 aa, Artificial sequence
  Synthetic polypeptide
PQPELPYPQ

SEQ ID NO: 29 - protein, 9 aa, Artificial sequence
  Synthetic polypeptide
FRPEQPYPQ

SEQ ID NO: 30 - protein, 9 aa, Artificial sequence
  Synthetic polypeptide
PQQSFPEQQ

SEQ ID NO: 31 - protein, 9 aa, Artificial sequence
  Synthetic polypeptide
IQPEQPAQL

SEQ ID NO: 32 - protein, 9 aa, Artificial sequence
  Synthetic polypeptide
QQPEQPYPQ

SEQ ID NO: 33 - protein, 9 aa, Artificial sequence
  Synthetic polypeptide
SQPEQEFPQ

SEQ ID NO: 34 - protein, 9 aa, Artificial sequence
  Synthetic polypeptide
PQPEQEFPQ

SEQ ID NO: 35 - protein, 9 aa, Artificial sequence
  Synthetic polypeptide
QQPEQPFPQ

SEQ ID NO: 36 - protein, 9 aa, Artificial sequence
  Synthetic polypeptide
PQPEQPFCQ

SEQ ID NO: 37 - protein, 9 aa, Artificial sequence
  Synthetic polypeptide
QQPFPEQPQ

SEQ ID NO: 38 - protein, 9 aa, Artificial sequence
  Synthetic polypeptide
PFPQPEQPF

SEQ ID NO: 39 - protein, 9 aa, Artificial sequence
  Synthetic polypeptide
PQPEQPFPW

SEQ ID NO: 40 - protein, 9 aa, Artificial sequence
  Synthetic polypeptide
PFSEQEQPV

SEQ ID NO: 41 - protein, 9 aa, Artificial sequence
  Synthetic polypeptide
FSQQQESPF

SEQ ID NO: 42 - protein, 9 aa, Artificial sequence
  Synthetic polypeptide
PFPQPEQPF

SEQ ID NO: 43 - protein, 9 aa, Artificial sequence
  Synthetic polypeptide
PQPEQPFPQ

SEQ ID NO: 44 - protein, 9 aa, Artificial sequence
  Synthetic polypeptide
PIPEQPQPY

SEQ ID NO: 45 - protein, 9 aa, Artificial sequence
  Synthetic polypeptide
PFPQPEQPF

SEQ ID NO: 46 - protein, 9 aa, Artificial sequence
  Synthetic polypeptide
PQPEQPFPQ

SEQ ID NO: 47 - protein, 9 aa, Artificial sequence
  Synthetic polypeptide
PYPEQEEPF

SEQ ID NO: 48 - protein, 9 aa, Artificial sequence
  Synthetic polypeptide
PYPEQEQPF

SEQ ID NO: 49 - protein, 9 aa, Artificial sequence
  Synthetic polypeptide
PFSEQEQPV

SEQ ID NO: 50 - protein, 9 aa, Artificial sequence
  Synthetic polypeptide
EGSFQPSQE

SEQ ID NO: 51 - protein, 9 aa, Artificial sequence
  Synthetic polypeptide
EQPQQPFPQ

SEQ ID NO: 52 - protein, 9 aa, Artificial sequence
  Synthetic polypeptide
EQPQQPYPE

SEQ ID NO: 53 - protein, 10 aa, Artificial sequence
  Synthetic polypeptide
QQGYYPTSPQ

SEQ ID NO: 54 - protein, 9 aa, Artificial sequence
  Synthetic polypeptide
EGSFQPSQE

SEQ ID NO: 55 - protein, 9 aa, Artificial sequence
  Synthetic polypeptide
PQQSFPEQE

SEQ ID NO: 56 - protein, 9 aa, Artificial sequence
  Synthetic polypeptide
QGYYPTSPQ

SEQ ID NO: 57 - protein, 9 aa, Artificial sequence
  Synthetic polypeptide
PQPQLPYPQ

SEQ ID NO: 58 - protein, 9 aa, Artificial sequence
  Synthetic polypeptide
FRPQQPYPQ

SEQ ID NO: 59 - protein, 9 aa, Artificial sequence
  Synthetic polypeptide
PQQSFPQQQ

SEQ ID NO: 60 - protein, 9 aa, Artificial sequence
  Synthetic polypeptide
IQPQQPAQL

SEQ ID NO: 61 - protein, 9 aa, Artificial sequence
  Synthetic polypeptide
QQPQQPYPQ

SEQ ID NO: 62 - protein, 9 aa, Artificial sequence
  Synthetic polypeptide
SQPQQQFPQ

SEQ ID NO: 63 - protein, 9 aa, Artificial sequence
  Synthetic polypeptide
PQPQQQFPQ

SEQ ID NO: 64 - protein, 9 aa, Artificial sequence
  Synthetic polypeptide
QQPQQPFPQ

SEQ ID NO: 65 - protein, 9 aa, Artificial sequence
  Synthetic polypeptide
PQPQQPFCQ

SEQ ID NO: 66 - protein, 9 aa, Artificial sequence
  Synthetic polypeptide
QQPFPQQPQ

SEQ ID NO: 67 - protein, 9 aa, Artificial sequence
  Synthetic polypeptide
PFPQPQQPF

SEQ ID NO: 68 - protein, 9 aa, Artificial sequence
  Synthetic polypeptide
PQPQQPFPW

SEQ ID NO: 69 - protein, 9 aa, Artificial sequence
  Synthetic polypeptide
PFSQQQQPV

SEQ ID NO: 70 - protein, 9 aa, Artificial sequence
  Synthetic polypeptide
FSQQQQSPF

SEQ ID NO: 71 - protein, 9 aa, Artificial sequence
  Synthetic polypeptide
PFPQPQQPF

SEQ ID NO: 72 - protein, 9 aa, Artificial sequence
  Synthetic polypeptide
PQPQQPFPQ

SEQ ID NO: 73 - protein, 9 aa, Artificial sequence
  Synthetic polypeptide
PIPQQPQPY

SEQ ID NO: 74 - protein, 9 aa, Artificial sequence
  Synthetic polypeptide
PFPQPQQPF

SEQ ID NO: 75 - protein, 9 aa, Artificial sequence
  Synthetic polypeptide
PQPQQPFPQ

SEQ ID NO: 76 - protein, 9 aa, Artificial sequence
  Synthetic polypeptide
PYPEQQEPF

SEQ ID NO: 77 - protein, 9 aa, Artificial sequence
  Synthetic polypeptide
PYPEQQQPF

SEQ ID NO: 78 - protein, 9 aa, Artificial sequence
  Synthetic polypeptide
PFSQQQQPV

SEQ ID NO: 79 - protein, 9 aa, Artificial sequence
  Synthetic polypeptide
QGSFQPSQQ

SEQ ID NO: 80 - protein, 9 aa, Artificial sequence
  Synthetic polypeptide
QQPQQPFPQ

SEQ ID NO: 81 - protein, 9 aa, Artificial sequence
  Synthetic polypeptide
QQPQQPYPQ

SEQ ID NO: 82 - protein, 10 aa, Artificial sequence
  Synthetic polypeptide
QQGYYPTSPQ

SEQ ID NO: 83 - protein, 9 aa, Artificial sequence
  Synthetic polypeptide
QGSFQPSQQ

SEQ ID NO: 84 - protein, 9 aa, Artificial sequence
  Synthetic polypeptide
PQQSFPQQQ

SEQ ID NO: 85 - protein, 9 aa, Artificial sequence
  Synthetic polypeptide
QGYYPTSPQ

SEQ ID NO: 86 - protein, 15 aa, Artificial sequence
  Synthetic polypeptide
LQPFPQPQLPYPQPQ

SEQ ID NO: 87 - protein, 14 aa, Artificial sequence
  Synthetic polypeptide
QPFPQPQQPFPWQP

SEQ ID NO: 88 - protein, 15 aa, Artificial sequence
  Synthetic polypeptide
PQQPIPQQPQPYPQQ

SEQ ID NO: 89 - protein, 7 aa, Artificial sequence
  Synthetic polypeptide
QPELPYP

SEQ ID NO: 90 - protein, 7 aa, Artificial sequence
  Synthetic polypeptide
PQPELPY

SEQ ID NO: 91 - protein, 7 aa, Artificial sequence
  Synthetic polypeptide
FPQPELP

SEQ ID NO: 92 - protein, 7 aa, Artificial sequence
  Synthetic polypeptide
PELPYPQ

SEQ ID NO: 93 - protein, 9 aa, Artificial sequence
  Synthetic polypeptide
FPQPELPYP

SEQ ID NO: 94 - protein, 9 aa, Artificial sequence
  Synthetic polypeptide
PQPDLPYPQ

SEQ ID NO: 95 - protein, 9 aa, Artificial sequence
  Synthetic polypeptide
PFPQPDQPF

SEQ ID NO: 96 - protein, 9 aa, Artificial sequence
  Synthetic polypeptide
PQPDQPFPW

SEQ ID NO: 97 - protein, 9 aa, Artificial sequence
  Synthetic polypeptide
PIPDQPQPY

SEQ ID NO: 98 - protein, 15 aa, Artificial sequence
  Synthetic polypeptide
LQPFPQPDLPYPQPQ

SEQ ID NO: 99 - protein, 14 aa, Artificial sequence
  Synthetic polypeptide
QPFPQPDQPFPWQP

SEQ ID NO: 100 - protein, 15 aa, Artificial sequence
  Synthetic polypeptide
PQQPIPDQPQPYPQQ

SEQ ID NO: 101 - protein, 7 aa, Artificial sequence
  Synthetic polypeptide
QPQLPYP

SEQ ID NO: 102 - protein, 7 aa, Artificial sequence
  Synthetic polypeptide
PQPQLPY

SEQ ID NO: 103 - protein, 7 aa, Artificial sequence
  Synthetic polypeptide
FPQPQLP

SEQ ID NO: 104 - protein, 7 aa, Artificial sequence
  Synthetic polypeptide
PQLPYPQ

SEQ ID NO: 105 - protein, 9 aa, Artificial sequence
  Synthetic polypeptide
FPQPQLPYP

(19) United States
(12) Patent Application Publication
(10) Pub. No.: US 2016/0041148 A1
(43) Pub. Date: Feb. 11, 2016

(54) PLACEBO-CONTROLLED GLUTEN CHALLENGE METHOD

(71) Applicant: IMMUSANT,INC., Cambridge, MA (US)

(72) Inventors: Robert P. Anderson, Shrewsbury, MA (US); Patrick Griffin, New York, NY (US)

(21) Appl. No.: 14/776,235

(22) PCT Filed: Mar. 14, 2014
(86) PCT No.: PCT/US14/27101

§ 371 Date: Sep. 14, 2015

Related U.S. Application Data
(60) Provisional application No. 61/785,295, filed on Mar. 14, 2013.

(51) Int. Cl. G01N 33/50

(52) U.S. Cl. CPC G01N 33/505; G01N 2800/065; G01N 2333/415; G01N 2333/57

(57) ABSTRACT

Provided herein are placebo-controlled methods for identifying Celiac disease in a subject, and related compositions and kits.

### CROSS-REFERENCE TO RELATED APPLICATIONS

This application claims the benefit of the filing date of U.S. Provisional Application No. 61/785,295, filed Mar. 14, 2013, the entire contents of which are incorporated by reference herein.

### BACKGROUND

Celiac disease (CD) is an autoimmune-like disorder of the small intestine that occurs in people of all ages. CD causes damage to the villi of the small intestine due to an inappropriate immune response to gluten peptides, leading to malabsorption and an increased risk of intestinal cancer. Correctly diagnosing CD is important in order to ensure that those affected by CD receive proper treatment.

### SUMMARY

As described herein, it has been discovered that administration of a gluten peptide-containing composition and a placebo composition in a placebo-controlled method (meaning the subject, and optionally the medical practitioner as well, are unaware of which composition contains a gluten peptide) can be used to determine if subjects have Celiac disease while reducing the concern that a placebo effect may result in a misdiagnosis or misidentification of the subject as having Celiac disease. Accordingly, aspects of the disclosure relate to methods for diagnosis or identification of Celiac disease in a subject, such as a subject having or suspected of having Celiac disease.

In one aspect, a method of assessing a T cell response in a subject, the method comprising: (a) measuring a T cell response in a first sample comprising T cells obtained from the subject after administration to the subject of a first composition comprising at least one gluten peptide; and (b) measuring a T cell response in a second sample comprising T cells obtained from the subject after administration of a placebo is provided.

In another aspect, a method of assessing a T cell response in a subject, the method comprising: (a) administering to the subject a first composition, the first composition comprising at least one gluten peptide; (b) measuring a T cell response in a first sample comprising T cells obtained from the subject after administration of the first composition; (c) administering to the subject a placebo; and (d) measuring a T cell response in a second sample comprising T cells obtained from the subject after administration of the placebo is provided.

In some embodiments of any one of the methods or compositions or kits provided herein, the first composition is administered to the subject before the placebo is administered to the subject. In some embodiments of any one of the methods or compositions or kits provided herein, the placebo is administered to the subject before the first composition is administered to the subject.

In some embodiments of any one of the methods or compositions or kits provided herein, the measuring of a T cell response in the first sample and the second sample are performed together in one assay.

In some embodiments of any one of the methods or compositions or kits provided herein, the sample comprises whole blood or peripheral blood mononuclear cells.

In some embodiments of any one of the methods or compositions or kits provided herein, the first composition is administered to the subject more than once. In some embodiments of any one of the methods or compositions or kits provided herein, the first composition is administered to the subject at least once a day for at least three days. In some embodiments of any one of the methods or compositions or kits provided herein, the first composition is administered to the subject at least three times a day for at least three days.

In some embodiments of any one of the methods or compositions or kits provided herein, the placebo is administered to the subject more than once. In some embodiments of any one of the methods or compositions or kits provided herein, the placebo is administered to the subject at least once a day for at least three days. In some embodiments of any one of the methods or compositions or kits provided herein, the placebo is administered to the subject at least three times a day for at least three days.

In some embodiments of any one of the methods or compositions or kits provided herein, the administration of the first composition and the placebo is oral administration.

In some embodiments of any one of the methods or compositions or kits provided herein, the first composition and the placebo are foodstuffs.

In some embodiments of any one of the methods or compositions or kits provided herein, the measuring of a T cell response in the first sample and the second sample comprises contacting the first and second samples with a second composition comprising at least one gluten peptide and measuring the level of at least one cytokine in the first and second samples. In some embodiments of any one of the methods or compositions or kits provided herein, the second composition contacted with the first sample and the second sample is the same composition.

In some embodiments of any one of the methods or compositions or kits provided herein, the at least one cytokine is IFN-γ. In some embodiments of any one of the methods or compositions or kits provided herein, the level of the at least one cytokine is measured with an enzyme-linked immunosorbent assay (ELISA). In some embodiments of any one of the methods or compositions or kits provided herein, the level of the at least one cytokine is measured with an enzyme-linked immunosorbent spot (ELISpot) assay.

In some embodiments of any one of the methods or compositions or kits provided herein, the first composition comprises at least one of: (i) a first peptide comprising the amino acid sequence PFPQPELPY (SEQ ID NO: 1), or a non-deamidated version thereof, and PQPELPYPQ (SEQ ID NO: 2), or a non-deamidated version thereof, (ii) a second peptide comprising the amino acid sequence PFPQPEQPF (SEQ ID NO: 3), or a non-deamidated version thereof, and PQPEQPFPW (SEQ ID NO: 4), or a non-deamidated version thereof, and (iii) a third peptide comprising the amino acid sequence PIPEQPQPY (SEQ ID NO: 5), or a non-deamidated version thereof.

In some embodiments of any one of the methods or compositions or kits provided herein, the second composition comprises at least one of: (i) a first peptide comprising the amino acid sequence PFPQPELPY (SEQ ID NO: 1), or a non-deamidated version thereof, and PQPELPYPQ (SEQ ID NO: 2), or a non-deamidated version thereof, (ii) a second peptide comprising the amino acid sequence PFPQPEQPF (SEQ ID NO: 3), or a non-deamidated version thereof, and PQPEQPFPW (SEQ ID NO: 4), or a non-deamidated version thereof, and (iii) a third peptide comprising the amino acid sequence PIPEQPQPY (SEQ ID NO: 5), or a non-deamidated version thereof.

In some embodiments of any one of the methods or compositions or kits provided herein, the first peptide comprises the amino acid sequence LQPFPQPQLPYPQPQ (SEQ ID NO: 86); the second peptide comprises the amino acid sequence QPFPQPQQPFPWQP (SEQ ID NO: 87); and/or the third peptide comprises the amino acid sequence PQQPIPQQPQPYPQQ (SEQ ID NO: 88).

In some embodiments of any one of the methods or compositions or kits provided herein, the second composition comprises at least one of: (i) a first peptide comprising the amino acid sequence PFPQPDLPY (SEQ ID NO: 27) and PQPDLPYPQ (SEQ ID NO: 94), (ii) a second peptide comprising the amino acid sequence PFPQPDQPF (SEQ ID NO: 95) and PQPDQPFPW (SEQ ID NO: 96), and (iii) a third peptide comprising the amino acid sequence PIPDQPQPY (SEQ ID NO: 97).

In some embodiments of any one of the methods or compositions or kits provided herein, the first peptide comprises the amino acid sequence LQPFPQPDLPYPQPQ (SEQ ID NO: 98), the second peptide comprises the amino acid sequence QPFPQPDQPFPWQP (SEQ ID NO: 99), and/or the third peptide comprises the amino acid sequence PQQPIPDQPQPYPQQ (SEQ ID NO: 100).

In some embodiments of any one of the methods or compositions or kits provided herein, the first peptide comprises the amino acid sequence LQPFPQPELPYPQPQ (SEQ ID NO: 6); the second peptide comprises the amino acid sequence QPFPQPEQPFPWQP (SEQ ID NO: 7); and/or the third peptide comprises the amino acid sequence PEQPIPEQPQPYPQQ (SEQ ID NO: 8).

In some embodiments of any one of the methods or compositions or kits provided herein, the first, second and/or third peptides comprise an N-terminal acetyl group or pyroglutamate group, and/or a C terminal amide group.

In some embodiments of any one of the methods or compositions or kits provided herein, the first peptide comprises the amino acid sequence ELQPFPQPELPYPQPQ (SEQ ID NO: 9), wherein the N-terminal E is a pyroglutamate; the second peptide comprises the amino acid sequence EQPFPQPEQPFPWQP (SEQ ID NO: 10), wherein the N-terminal E is a pyroglutamate; and the third peptide comprises the amino acid sequence EPEQPIPEQPQPYPQQ (SEQ ID NO: 11), wherein the N-terminal E is a pyroglutamate.

In some embodiments of any one of the methods or compositions or kits provided herein, the first peptide consists of the amino acid sequence ELQPFPQPELPYPQPQ (SEQ ID NO: 9), wherein the N-terminal E is a pyroglutamate; the second peptide consists of the amino acid sequence EQPFPQPEQPFPWQP (SEQ ID NO: 10), wherein the N-terminal E is a pyroglutamate; and the third peptide consists of the amino acid sequence EPEQPIPEQPQPYPQQ (SEQ ID NO: 11), wherein the N-terminal E is a pyroglutamate.

In some embodiments of any one of the methods or compositions or kits provided herein, the first peptide consists of the amino acid sequence ELQPFPQPELPYPQPQ (SEQ ID NO: 9), wherein the N-terminal E is a pyroglutamate, and wherein the first peptide contains a C-terminal amide group; the second peptide consists of the amino acid sequence EQPFPQPEQPFPWQP (SEQ ID NO: 10), wherein the N-terminal E is a pyroglutamate, and wherein the second peptide contains a C-terminal amide group; and/or the third peptide consists of the amino acid sequence EPEQPIPEQPQPYPQQ (SEQ ID NO: 11), wherein the N-terminal E is a pyroglutamate, and wherein the third peptide contains a C-terminal amide group.

In some embodiments of any one of the methods or compositions or kits provided herein, the first, second, and/or third peptide are each independently 8-50 amino acids in length.

In some embodiments of any one of the methods or compositions or kits provided herein, the method comprises comparing the T cell response measured in the first sample with a control T cell response and comparing the T cell response measured in the second sample with the control T cell response. In some embodiments of any one of the methods or compositions or kits provided herein, the level of the control T cell response is any of the levels provided herein including in the Examples.

In some embodiments of any one of the methods or compositions or kits provided herein, the method further comprises comparing the T cell response measured in the first sample with the T cell response measured in the second sample to identify or aid in identifying the subject as having Celiac disease or as being in need of other testing if the T cell response measured in the first sample is elevated compared to the T cell response measured in the second sample, or to identify or aid in identifying the subject as not having or unlikely of having Celiac disease or as not having or unlikely of being in need of other testing if the T cell response measured in the first sample is substantially the same or decreased compared to the T cell response measured in the second sample.

In some embodiments of any one of the methods or compositions or kits provided herein, the method further comprises: treating the subject with a therapy if the subject is identified as having Celiac disease or recommending or providing information about a therapy to the subject.

In some embodiments of any one of the methods or compositions or kits provided herein, the therapy is a gluten-free diet.

In some embodiments of any one of the methods or compositions or kits provided herein, the method further comprises performing another test on the subject prior to or after the steps of the method, preferably, in some embodiments, performing a serology and/or genotyping assay. In some embodiments of any one of the methods or compositions or kits provided herein, the performing a serology and/or genotyping assay occurs prior to all of the steps recited in the method. In some embodiments of any one of the methods or compositions or kits provided herein, the performing a serology and/or genotyping assay occurs after all of the steps recited in the method.

In some embodiments of any one of the methods or compositions or kits provided herein, the subject is suspected of having Celiac disease. In some embodiments of any one of the methods or compositions or kits provided herein, the subject is HLA-DQ2.5 positive.

In some embodiments of any one of the methods or compositions or kits provided herein, the first sample is obtained from the subject at least one day after administration of the first composition and the second sample is obtained from the subject at least one day after administration of the placebo.

In some embodiments of any one of the methods or compositions or kits provided herein, the first sample is obtained from the subject at least six days after administration of the first composition and the second sample is obtained from the subject at least six days after administration of the placebo.

In another aspect, a kit comprising a composition comprising a gluten peptide and a placebo is provided.

In some embodiments of any one of the methods or compositions or kits provided herein, the composition and the placebo are foodstuffs.

In some embodiments of any one of the methods or compositions or kits provided herein, the composition comprises at least one of a wheat gluten, a barley hordein, and a rye secalin. In some embodiments of any one of the methods or compositions or kits provided herein, the composition comprises at least two of a wheat gluten, a barley hordein, and a rye secalin. In some embodiments of any one of the methods or compositions or kits provided herein, the composition comprises a wheat gluten, a barley hordein, and a rye secalin.

In some embodiments of any one of the methods or compositions or kits provided herein, the composition comprises a container, such as a vial or tube, for whole blood.

In some embodiments of any one of the methods or compositions or kits provided herein, one or more gluten peptides are dried on the wall of the container for whole blood.

In some embodiments of any one of the methods or compositions or kits provided herein, one or more gluten peptides are in solution or lyophilized in a separate container.

In some embodiments of any one of the methods or compositions or kits provided herein, the one or more gluten peptides are as defined in any of the claims provided herein or as described elsewhere herein.

In some embodiments of any one of the methods or compositions or kits provided herein, the kit further comprises an anticoagulant.

In some embodiments of any one of the methods or compositions or kits provided herein, the container for whole blood and/or other container are present in duplicate or triplicate.

In some embodiments of any one of the methods or compositions or kits provided herein, the kit further comprises a negative control container, such as a vial or tube.

In some embodiments of any one of the methods or compositions or kits provided herein, the kit further comprises a positive control container, such as a vial or tube.

In some embodiments of any one of the methods or compositions or kits provided herein, the negative and/or positive control container(s) are present in duplicate or triplicate.

In some embodiments of any one of the methods or compositions or kits provided herein, the amount of each of the gluten peptides is any of the amounts provided herein including in the Examples.

In some embodiments, any one of the compositions or kits may be for use in any one of the methods provided.

In some embodiments, any one of the methods can include the use of any one of the compositions or kits provided.

### BRIEF DESCRIPTION OF THE DRAWINGS

FIG. 1 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

FIG. 2 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

FIG. 3 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

FIG. 4 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

FIG. 5 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

FIG. 6 provides two graphs depicting the interferon-γ response over the course of the placebo-controlled gluten challenge in a patient (top) and the interferon-γ response normalized to the “nil” tube for the patient (bottom). For the top graph, the y-axis is IFN-γ (pg/mL) and the x-axis values are from left to right, D0, D1, D6, D8, D13, D21. For the bottom graph, the y-axis is Signal:Nil Ratio and the x-axis values are from left to right, D0, D1, D6, D8, D13, D21.

### DETAILED DESCRIPTION

Celiac disease (CD, also sometimes referred to as Cœliac disease, C(o)eliac sprue, non-tropical sprue, endemic sprue, gluten enteropathy, etc.) is defined by the presence of small intestinal inflammation that improves or normalizes with exclusion of dietary gluten derived from foods including wheat, barley and rye. Celiac disease is one of a cluster of diseases associated with autoantibody production (IgA specific for transglutaminase-2) and T-cell mediated organ-specific immunopathology that are strongly associated with HLA-DR3-DQ2 and DR4-DQ8 haplotypes. In celiac disease peptides derived from an exogenous antigen, dietary gluten, are recognized by pathogenic T cells, such as CD4⁺ T cells. Celiac disease occurs in people of all ages after gluten has been included in the diet i.e. middle infancy onward. Celiac disease affects approximately 1% of people in Europe and North America. In many of those affected, Celiac disease is unrecognized, but this clinical oversight is now being rectified with greater clinical awareness.

Celiac disease occurs in genetically susceptible individuals who possess either HLA-DQ2 encoded by HLA-DQAl *05 and HLA-DQBl *02 (accounting for about 90% of individuals), variants of HLA-DQ2, or HLA-DQ8. Without wishing to be bound by theory, it is believed that these individuals mount an inappropriate HLA-DQ2- and/or DQ8-restricted CD4⁺ T cell-mediated immune response to peptides derived from the aqueous-insoluble proteins of wheat flour, gluten, and related proteins in rye and barley.

Currently, reversal of an incorrect diagnosis of Celiac disease requires HLA-DQ genetic testing and, in genetically-susceptible individuals, extended gluten challenge and demonstration of normal small bowel histology while ingesting a normal gluten-containing diet (Hadithi M et al. Ann Intern Med. 2007, Kumar P. et al. Gut 1979, Husby S, et al. J Pediatr Gastroenterol Nutr. 2012). HLA-DQ genotype is an exceptionally powerful negative predictor of Celiac disease in patients, but more than half of Caucasians possess HLA-DQ2.5, DQ8, or DQ2.2 (Karell K, et al. Hum Immunol 2003). This renders the positive predictive value of genotyping alone weak as many individuals possess HLA-DQ2.5, DQ8, or DQ2.2 but do not have Celiac disease, which would result in a high rate of “false positive” diagnoses if used alone.

About half of the patients with a “false positive” diagnosis would be expected to possess HLA DQ2.5, DQ2.2, or DQ8, and cannot be distinguished from patients with Celiac disease by genetic testing (Koskinen S et al. Immunogenetics 2009). In patients with Celiac disease following a strict gluten-free diet, but not in healthy non-Celiac disease subjects, gluten challenge, for example for three days (Day-1 to Day-3), allows CD4⁺ T cells specific for immunodominant gluten peptides to be detected on Day-6 (Anderson R P et al. Nature Med 2000). However, a “placebo-gluten” challenge has not previously been performed to confirm that CD4⁺ T cell responses are not related to a placebo effect.

As disclosed herein, testing was performed to determine whether CD4⁺ T cells specific for immunodominant gluten peptides derived from wheat, barley and rye prolamins (plant storage proteins with a characteristically high proline content, which include gliadin, hordein, and secalin) could be detected in whole blood six days after commencing eating a gluten-containing food, but not after eating an indistinguishable “placebo” food in patients following a strict gluten-free diet diagnosed with Celiac disease according to established criteria (Revised criteria for diagnosis of coeliac disease. Report of Working Group of European Society of Paediatric Gastroenterology and Nutrition. Arch Dis Child 1990; 65:909-11). It was determined that ex vivo whole blood interferon-γ (IFN-γ) release stimulated by a mixture of gluten peptides in most instances was restricted to subjects with Celiac disease after gluten challenge but not after placebo challenge.

Accordingly, aspects of the disclosure relate to placebo-controlled methods for identifying Celiac disease in subjects having or suspected of having Celiac disease.

### Diagnostic Methods

One aspect of the disclosure relates to placebo-controlled methods for subjects, such as subjects having or suspected of having Celiac disease.

In some embodiments, the method is a method of assessing a T cell response in a subject, the method comprising: (a) measuring a T cell response in a first sample comprising T cells obtained from the subject after administration to the subject a first composition comprising at least one gluten peptide as described herein; and (b) measuring a T cell response in a second sample comprising T cells obtained from the subject after administration of a placebo as described herein. Without wishing to be bound by theory, it is believed that the gluten peptide serves as the active component causing the activation and/or mobilization of CD4⁺ T cells in a subject who has Celiac disease. Thus, in some embodiments, the T cell or T cell response referred to in any of the methods provided is a CD4+ T cell or CD4+ T cell response. In some embodiments, the method comprises: (a) determining a T cell response in a first sample comprising T cells obtained from the subject after administration to the subject a first composition comprising at least one gluten peptide as described herein; and (b) determining a T cell response in a second sample comprising T cells obtained from the subject after administration of a placebo as described herein. In some embodiments, the method comprises: (a) administering to the subject a first composition, the first composition comprising at least one gluten peptide as described herein; (b) measuring a T cell response in a first sample comprising T cells obtained from the subject after administration of the first composition; (c) administering to the subject a placebo as described herein; and (d) measuring a T cell response in a second sample comprising T cells obtained from the subject after administration of the placebo. In some embodiments, the subject has or is suspected of having Celiac disease.

It is to be understood that the order of the steps may vary, as long as the first and second samples are collected after the administration of the first composition and the placebo, respectively. Accordingly, in some embodiments, the first composition is administered to the subject before the placebo is administered to the subject. In other embodiments, the placebo is administered to the subject before the first composition is administered to the subject. The order of administration is meant to be blinded to the subject (“single-blinded”) or to the subject and the medical practitioner or other person skilled in the art involved in measuring a T cell response and general monitoring of the subject (“double-blinded”). In some embodiments, the measuring of a T cell response in the first sample and the measuring of a T cell response in the second sample are performed at the same time as part of the same assay (e.g., after administration of both the first composition and the placebo). In some embodiments, the first sample is obtained from the subject at least one day, e.g., 1, 2, 3, 4, 5, 6, 7, 8, 9, 10, 11, 12, 13 or 14 days, after administration of the first composition and the second sample is obtained from the subject at least one day, e.g., 1, 2, 3, 4, 5, 6, 7, 8, 9, 10, 11, 12, 13 or 14 days, after administration of the placebo. In some embodiments, the first sample is obtained from the subject 6 days after administration of the first composition and the second sample is obtained from the subject 6 days after administration of the placebo. In some embodiments, the methods include obtaining a baseline sample, for example, in methods where the placebo is administered within a month after an active challenge.

In some embodiments, the method further comprises comparing the T cell response measured in the first sample with the T cell response measured in the second sample to identify or aid in identifying the subject as having Celiac disease or as being in need of other testing if the T cell response measured in the first sample is elevated compared to the T cell response measured in the second sample, or to identify or aid in identifying the subject as not having or unlikely of having Celiac disease or as not having or unlikely of being in need of other testing if the T cell response measured in the first sample is substantially the same or decreased compared to the T cell response measured in the second sample. For example, if the subject has an elevated T cell response to the first composition compared to the placebo, the subject can be identified as having Celiac disease or as being in need of other testing. In a second example, if the subject has the same or a decreased T cell response to the first composition compared to the placebo, the subject can be identified as not having or unlikely of having Celiac disease or as not having or unlikely of being in need of other testing. In this second example, if the T cell response to the first composition and to the placebo is increased relative to a negative control T cell response, this may be indicative of the placebo effect. The placebo effect is described in detail herein. The comparisons can be done according to statistical methods. Such methods are well known to those of ordinary skill in the art. In some embodiments, “substantially the same” means not statistically significantly different.

In some embodiments, the method further comprises identifying or aiding in identifying the subject as having Celiac disease or as being in need of other testing if the T cell response measured in the first sample is elevated compared to the T cell response measured in the second sample, or identifying or aiding in identifying the subject as not having or unlikely of having Celiac disease or as not having or unlikely of being in need of other testing if the T cell response measured in the first sample is substantially the same or decreased compared to the T cell response measured in the second sample. In some embodiments, the method further comprises diagnosing the subject as having Celiac disease or as in need of other testing if the T cell response measured in the first sample is elevated compared to the T cell response measured in the second sample, or diagnosing the subject as not having or unlikely of having Celiac disease or as not having or unlikely of being in need of other testing if the T cell response measured in the first sample is substantially the same or decreased compared to the T cell response measured in the second sample. In some embodiments, the method further comprises indicating that the subject has Celiac disease or is in need of other testing if the T cell response measured in the first sample is elevated compared to the T cell response measured in the second sample or indicating that the subject does not have or is unlikely to have Celiac disease or is not in, or is unlikely to be in, need of other testing if the T cell response measured in the first sample is substantially the same or decreased compared to the T cell response measured in the second sample.

In some embodiments, the method further comprises performing other testing if the subject is identified or diagnosed as in need of other testing. Any method of other testing as described herein is contemplated. In some embodiments, the other testing comprises a serology test, genotyping, and/or an intestinal biopsy.

In some embodiments, the method further comprises recommending a gluten-free diet and/or providing information in regard thereto to the subject. In some embodiments, the method further comprises treating or providing information in regard to a treatment to the subject. In some embodiments, the method further comprises administering a treatment to the subject. Suitable treatments are described herein. In some embodiments, the treatment is a composition comprising a gluten peptide as described herein. In some embodiments, the treatment comprises a gluten-free diet.

In some embodiments, the method further comprises obtaining the first and second sample comprising T cells from the subject before measuring the T cell response in the first and second sample.

It is to be understood that “measuring” as used herein involves a physical step, e.g., an assay, and is not meant to encompass only a mental step or abstract idea. Assays appropriate for measuring a T cell response are described herein or are otherwise known in the art.

It is to be understood that “diagnosing” or making a determination about a subject's Celiac status and/or a subject's need for further testing can be performed by a clinician and/or according to standard clinical practices, in some embodiments.

### Placebo

Aspects of the disclosure relate to administration of a placebo to a subject, such as a subject having or suspected of having Celiac disease, to reduce the risk of generating a false positive result due to a phenomenon known as the placebo effect. The placebo effect occurs when a subject has a perceived or actual improvement in or worsening of a medical condition following administration of an inert or otherwise medically ineffectual treatment (see, e.g., Lanotte M, Lopiano L, Torre E, Bergamasco B, Colloca L, Benedetti F (November 2005). “Expectation enhances autonomic responses to stimulation of the human subthalamic limbic region”. Brain, Behavior, and Immunity 19 (6): 500-9; Gensini G F, Conti A A, Conti A (April 2005). “Past and present of what will please the lord: an updated history of the concept of placebo”. Minerva Med 96 (2): 121-4; Kaptchuk T J, Friedlander E, Kelley J M, et al. (2010). Boutron, Isabelle. ed. “Placebos without Deception: A Randomized Controlled Trial in Irritable Bowel Syndrome”. PLoS ONE 5 (12): e15591; Hróbjartsson A, Norup M (June 2003). “The use of placebo interventions in medical practice—a national questionnaire survey of Danish clinicians”. Evaluation & the Health Professions 26 (2): 153-65; Beecher, H. K. (1955). “The powerful placebo”. Journal of the American Medical Association 159 (17): 1602-1606; Moerman D E, Jonas W B (2002). “Deconstructing the placebo effect and finding the meaning response”. Ann Intern Med. 136 (6): 471-6; Ho K H, Hashish I, Salmon P, Freeman R, Harvey W (1988). “Reduction of post-operative swelling by a placebo effect”. Journal of Psychosomatic Research 32 (2): 197-205; and Hashish I, Harvey W, Harris M (February 1986). “Anti-inflammatory effects of ultrasound therapy: evidence for a major placebo effect”. British Journal of Rheumatology 25 (1): 77-81). In the present disclosure, it is desirable to distinguish between a subject who has a CD4⁺ T cell response after administration of a composition comprising a gluten peptide because the subject has Celiac disease (a disease-mediated effect) versus a subject who has a CD4⁺ T cell response after administration of a composition comprising a gluten peptide because of a placebo effect, for example, the subject believes or is convinced that they have Celiac disease. A subject who does not have Celiac disease is expected to not have a CD4⁺ T cell response after administration of a composition comprising a gluten peptide, as the composition would be non-reactive in such a subject. The presence of a CD4⁺ T cell response in a subject who does not have Celiac disease may indicate a placebo effect. Administration of a placebo is expected to aid in identifying subjects experiencing the placebo effect, as a subject experiencing the placebo effect should have a CD4⁺ T cell response both to the composition comprising a gluten peptide and to the placebo. This should allow for subjects who have Celiac disease to be distinguished from subjects who do not have Celiac disease but are experiencing a placebo effect.

Any appropriate placebo is contemplated. A preferred placebo is nearly or entirely indistinguishable from the composition causing the desired medical effect. The desired effect herein, in preferred embodiments, is activation and/or mobilization of CD4⁺ T cells in a subject who has Celiac disease after administration of a composition comprising a gluten peptide. Without wishing to be bound by theory, it is believed that the gluten peptide serves as the active component causing the activation and/or mobilization of CD4⁺ T cells in a subject who has Celiac disease. The activation and/or mobilization of CD4⁺ T cells can be measured in a sample comprising T cells from the subject as described herein. Accordingly, in some embodiments, the placebo does not contain a gluten peptide (or is not in amount that causes what would be considered a positive T cell response) or is “gluten-free”. A placebo can be determined to be gluten-free using standard definitions (see, e.g., Codex Alimentarius as measured by accepted gluten food tests such as R5-ELISA <20 ppm or no detectable gluten TGA Australia). Depending on the route of administration, the degree of alteration and disguise of the placebo may vary. In some embodiments, the placebo contains the same components as the composition but does not contain a gluten peptide (or an amount that causes what would be considered a positive T cell response).

In some embodiments, the gluten peptide composition and the placebo are administered orally (e.g., as foodstuffs). In such embodiments, the placebo and the composition should have a similar taste, texture and appearance such that a subject cannot distinguish between the two while consuming each foodstuff. For example, the composition comprising a gluten peptide may be a foodstuff (such a cookie, muffin, or bread) containing wheat gluten, barley hordein, and/or rye secalin. The corresponding placebo foodstuff in some embodiments does not contain any of wheat gluten, barley hordein, and rye secalin. It is expected that, generally, omission of wheat, barley, and rye will alter the taste, texture and/or appearance of the placebo foodstuff. To bring the taste, texture and/or appearance of the placebo closer to that of the foodstuff comprising a gluten peptide, the placebo may comprise additional components or comprise alterations or omissions of components found in the gluten-peptide-containing foodstuff. Such additional components include, e.g., fillers, sweetening agents, flavoring agents, coloring agents, thickening agents (e.g., xantam gum, arrowroot, or guar gum), and preserving agents. Such additional components should ideally not comprise a gluten peptide. Exemplary fillers include, but are not limited to, flours that have no gluten peptides such as Almond flour, Amaranth flour, Buckwheat flour, Chestnut flour, Coconut flour, Corn flour, Millet flour, Montina® flour, Quinoa flour, Rice flour, Sorghum flour, Teff flour, Garbanzo Bean flour, Soy flour, Potato flour, Tapioca flour, and combinations thereof. Accordingly, in some embodiments, the placebo does not contain a gluten peptide; and comprises at least one additional component and/or excludes or alters at least one component of the composition comprising a gluten peptide.

### Placebo-Controlled Gluten Challenge

In some embodiments, methods provided herein comprise a placebo-controlled gluten challenge and/or measuring a T cell response in one or more samples obtained from a subject before, during, or after a placebo-controlled gluten challenge. As defined herein, a placebo-controlled gluten challenge comprises administration of two compositions to a subject: a composition comprising a gluten peptide as defined herein (i.e., the “active composition”) and a placebo as described herein (i.e., the “inactive composition”). The identity of the two compositions is to be concealed from the subject (“single-blinded”) or the subject and the individual involved in measuring a T cell response or monitoring the subject (“double-blinded”).

In some embodiments, the composition comprising a gluten peptide is administered to the subject for a defined period of time in order to activate gluten-reactive CD4⁺ T cells and/or mobilize such CD4⁺ T cells in the subject. The composition comprising a gluten peptide may be administered using the methods of gluten challenge known in the art. Currently, the standard gluten challenge lasts for several weeks (e.g., 4 weeks or more) and involves high doses of orally administered gluten peptides (e.g., 5-30 grams of gluten daily usually in the form of one or more slices of wheat bread or other baked goods that include gluten). Some studies suggest that a shorter challenge, e.g., through use of as little as 3 days of oral gluten challenge, is sufficient to activate and/or mobilize gluten-reactive CD4⁺ T cells (Anderson R, van Heel D, Tye-Din J, Barnardo M, Salio M, Jewell D, and Hill A. T cells in peripheral blood after gluten challenge in coeliac disease. Gut 2005; 54; 1217-1223; and In vivo antigen challenge in Celiac disease identifies a single transglutaminase-modified peptide as the dominant A-gliadin T cell epitope. Anderson R, Degano P, Godkin A, Jewell D, and Hill A. Nature Medicine. 2000; 6(3):337-342.). Any such administration of a composition comprising a gluten peptide that is capable of activating gluten-reactive CD4⁺ T cells and and/or mobilizing such CD4⁺ T cells into blood is contemplated herein. The administration may occur more than once, e.g., two or more times daily, daily, bi-weekly, or weekly. In some embodiments, administration is daily for 1, 2, 3, 4, 5, 6, 7, 8, 9, 10, 11, 12, 13, 14 or more days, or 1, 2, 3, 4, 5, 6, 7, 8 or more weeks. In some embodiments, administration is daily for 3 days. In some embodiments, administration is at least once daily (i.e., 1, 2, 3, 4, 5 or more times daily) for 1, 2, 3, 4, 5, 6, 7, 8, 9, 10, 11, 12, 13, 14 or more days, or 1, 2, 3, 4, 5, 6, 7, 8 or more weeks. In some embodiments, administration is at least once daily (i.e., 1, 2, 3, 4, 5 or more times daily) for 3 days. In some embodiments, the composition comprising a gluten peptide is administered to the subject three times a day for three days.

Administration of the placebo to the subject may be for the same number of times and for the same number of days as the composition comprising a gluten peptide. However, the placebo may be administered using a different dosage schedule than the composition comprising a gluten peptide. For example, the administration of the placebo may occur more than once, e.g., two or more times daily, daily, bi-weekly, or weekly. In some embodiments, placebo administration is daily for 1, 2, 3, 4, 5, 6, 7, 8, 9, 10, 11, 12, 13, 14 or more days, or 1, 2, 3, 4, 5, 6, 7, 8 or more weeks. In some embodiments, placebo administration is daily for 3 days. In some embodiments, placebo administration is at least once daily (i.e., 1, 2, 3, 4, 5 or more times daily) for 1, 2, 3, 4, 5, 6, 7, 8, 9, 10, 11, 12, 13, 14 or more days, or 1, 2, 3, 4, 5, 6, 7, 8 or more weeks. In some embodiments, administration is at least once daily (i.e., 1, 2, 3, 4, 5 or more times daily) for 3 days. In some embodiments, the placebo is administered to the subject three times a day for three days.

The order of administration of the composition comprising a gluten peptide and the placebo may be random. For example, the composition comprising a gluten peptide may be administered first and the placebo administered second for one subject, and the order may be reversed (i.e., placebo first and composition comprising a gluten peptide second) for another subject.

A delay between administration of the composition comprising a gluten peptide and the placebo is also contemplated herein. Such a delay may be desirable as it may allow clearing of gluten-reactive CD4⁺ T cells from the blood after administration of the composition comprising a gluten peptide or administration of the placebo in order to prevent skewing of the results due to the presence of gluten-reactive CD4⁺ T cells from a previous administration of a composition or placebo. In some embodiments, administration of the composition comprising a gluten peptide and the placebo occur, e.g., 1, 2, 3, 4, 5, 6, 7, 8, 9, 10, 11, 12, 13, 14 or more days apart, or 1, 2, 3, 4, 5, 6, 7, 8, 9, 10 or more weeks apart. In some embodiments, administration of the composition comprising a gluten peptide and the placebo occur 21, 20, 19, 18, 17, 16, 15, 14, 13, 12, 11, 10, 9, 8, 7, 6, 5, 4, 3, 2 or 1 day(s) apart.

Administration of the composition comprising a gluten peptide and the placebo may be self-administration by the subject or administration by a qualified individual, e.g., a medical practitioner such as a doctor or nurse. Such administration may be through any method known in the art. Compositions suitable for each administration route are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005). In some embodiments, administration of the composition comprising a gluten peptide and the placebo is oral administration.

Suitable forms of oral administration include foodstuffs (e.g., baked goods such as breads, cookies, muffins, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions and such compositions may contain one or more agents such as sweetening agents, flavoring agents, coloring agents and preserving agents in order to provide pharmaceutically elegant and palatable preparations.

### T Cell Responses and Measurement Thereof

Aspects of the disclosure relate to a determination or measurement of a T cell response in a sample comprising T cells from a subject, such as a subject having or suspected of having Celiac disease. In some embodiments, a first composition comprising a gluten peptide as described herein is administered to a subject and is capable of activating a CD4⁺ T cell in a subject, e.g., a subject with Celiac disease. The term “activate” or “activating” or “activation” in relation to a CD4⁺ T cell refers to the presentation by an MHC molecule of an epitope on one cell to an appropriate T cell receptor on a second CD4⁺ T cell, together with binding of a co-stimulatory molecule by the CD4⁺ T cell, thereby eliciting a “T cell response”, in this example, a CD4⁺ T cell response. Such a T cell response can be measured ex vivo, e.g., by measuring a T cell response in a sample comprising T cells from the subject.

As described herein, an elevated T cell response, such as an elevated CD4⁺ T cell response, from a sample comprising T cells from a subject after administration of a composition comprising a gluten peptide to the subject compared to a T cell response from a sample comprising T cells from a subject after administration of a placebo can correlate with the presence or absence of Celiac disease in the subject. Accordingly, aspects of the disclosure relate to methods that comprise determining or measuring a T cell response in a sample comprising T cells from a subject, e.g., having or suspected of having Celiac disease.

In some embodiments, measuring a T cell response in a sample comprising T cells from a subject comprises contacting the sample with a second composition comprising at least one gluten peptide as described herein. For example, whole blood or PBMCs obtained from a subject who has been exposed to a gluten peptide (e.g., by administration of a first composition comprising a gluten peptide) may be contacted with the second composition in order to stimulate T cells in the whole blood sample.

Measuring a T cell response can be accomplished using any assay known in the art (see, e.g., Molecular Cloning: A Laboratory Manual, J. Sambrook, et al., eds., Third Edition, Cold Spring Harbor Laboratory Press, Cold Spring Harbor, N. Y., 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York). In some embodiments, measuring a T cell response comprises an MHC Class II tetramer assay, such as flow cytometry with MHC Class II tetramer staining (see, e.g., Raki M, Fallang L E, Brottveit M, Bergseng E, Quarsten H, Lundin K E, Sollid L M: Tetramer visualization of gut-homing gluten-specific T cells in the peripheral blood of Celiac disease patients. Proceedings of the National Academy of Sciences of the United States of America 2007; Anderson R P, van Heel D A, Tye-Din J A, Barnardo M, Salio M, Jewell D P, Hill A V: T cells in peripheral blood after gluten challenge in coeliac disease. Gut 2005, 54(9):1217-1223; Brottveit M, Raki M, Bergseng E, Fallang L E, Simonsen B, Lovik A, Larsen S, Loberg E M, Jahnsen F L, Sollid L M et al: Assessing possible Celiac disease by an HLA-DQ2-gliadin Tetramer Test. The American journal of gastroenterology 2011, 106(7):1318-1324; and Anderson R P, Degano P, Godkin A J, Jewell D P, Hill A V: In vivo antigen challenge in Celiac disease identifies a single transglutaminase-modified peptide as the dominant A-gliadin T cell epitope. Nature Medicine 2000, 6(3):337-342).

In some embodiments, measuring a T cell response in a sample comprising T cells from a subject comprises measuring a level of at least one cytokine in the sample. In some embodiments, measuring a T cell response in a sample comprising T cells from a subject comprises contacting the sample with a second composition comprising at least one gluten peptide as described herein and measuring a level of at least one cytokine in the sample. In some embodiments, the at least one cytokine is at least one pro-inflammatory cytokine such as IL-2, IFN-γ, IL-4, IL-5, IP-10, IL-13, and IL-17, or chemokines such as MCP-1 and GM-CSF released, e.g., by monocytes or granulocytes, as a result of secretion of these cytokines. In some embodiments, the at least one cytokine is IFN-γ.

Interferon-γ (IFN-γ, also called IFNG, IFG, and IFI) is a dimerized soluble cytokine of the type II class of interferons. IFN-γ typically binds to a heterodimeric receptor consisting of Interferon γ receptor 1 (IFNGR1) and Interferon γ receptor 2 (IFNGR2). IFN-γ can also bind to the glycosaminoglycan heparan sulfate (HS). IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops in a subject. In humans, the IFN-γ protein is encoded by the IFNG gene. The Genbank number for the human IFNG gene is 3458. Exemplary Genbank mRNA transcript IDs and protein IDs for IFN-γ are NM_{—}000619.2 and NP_{—}000610.2, respectively.

In some embodiments, measuring a T cell response comprises measuring a level of at least one cytokine. Levels of at least one cytokine include levels of cytokine RNA, e.g., mRNA, and/or levels of cytokine protein. In a preferred embodiment, levels of the at least one cytokine are protein levels.

Assays for detecting cytokine RNA include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine, e.g., IFN-γ, the sequence(s) being identifiable using the Genbank IDs described herein or as otherwise known in the art.

Assays for detecting protein levels include, but are not limited to, immunoassays (also referred to herein as immune-based or immuno-based assays, e.g., Western blot, ELISA, and ELISpot assays), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine, e.g., IFN-γ, the sequence(s) being identifiable using the Genbank IDs described herein or as otherwise known in the art. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

In a preferred embodiment, measuring a level of at least one cytokine comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939,281, 6,410,252, and 7,575,870; Czerkinsky C, Nilsson L, Nygren H, Ouchterlony O, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51 (12): 2415-8).

An exemplary ELISA involves at least one binding partner, e.g., an antibody or antigen-binding fragment thereof, with specificity for the at least one cytokine, e.g., IFN-γ. The sample with an unknown amount of the at least one cytokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the antigen is immobilized, the binding partner for the at least one cytokine is added, forming a complex with the immobilized at least one cytokine. The binding partner can be attached to a detectable label as described herein (e.g., a fluorophor or an enzyme), or can itself be detected by an agent that recognizes the at least one cytokine binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

An exemplary ELISpot assay involves a binding agent for the at least one cytokine (e.g., an anti-IFN-γ) that is coated aseptically onto a PVDF (polyvinylidene fluoride)-backed microplate. Cells of interest (e.g., peripheral blood mononuclear cells) are plated out at varying densities, along with antigen (e.g., a gluten peptide as described herein), and allowed to incubate for a period of time (e.g., about 24 hours). The at least one cytokine secreted by activated cells is captured locally by the binding partner for the at least one cytokine on the high surface area PVDF membrane. After the at least one cytokine is immobilized, a second binding partner for the at least one cytokine is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of at least one cytokine is measured using an ELISA. As an exemplary method, at least one gluten peptide as defined herein is dried onto the inner wall of a blood collection tube. A negative control tube containing no antigen is provided. A positive control tube containing a mitogen is also provided. Blood from a subject is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of at least one cytokine (e.g., IFN-γ) present in the plasma. A standard ELISA assay as described above can then be used to detect the levels of the at least one cytokine present in each plasma sample. In some embodiments, a T cell response measurement in a sample obtained after administration of a composition comprising a gluten peptide to the subject is detected using any of the methods above or any other appropriate method and is then compared to a control T cell response, e.g., a T cell response measurement in a sample obtained after administration of a placebo as described herein. In some embodiments, a control T cell response is measured using any of the methods above or any other appropriate methods. In some embodiments, the same method is used to measure a T cell response in the sample of the subject and the sample obtained after administration of a placebo.

In some embodiments, a second control T cell response is contemplated. In some embodiments, the second control T cell response is a negative control T cell response. Exemplary negative controls include, but are not limited to, a T cell response in a sample that has been contacted with a non-T cell-activating peptide (e.g., a peptide not recognized by T cells present in a sample from a subject), such as a non-CD4⁺-T cell-activating peptide, or a T cell response in sample that has not been contacted with a T cell-activating peptide (e.g., contacting the sample with a saline solution containing no peptides), such as a CD4⁺ T cell-activating peptide. Another exemplary second control T cell response can be obtained using a sample from the subject before administration of both the placebo and the composition, such that a baseline T cell response can be established. Such a second control T cell response can be measured using any of the methods above or any other appropriate methods.

### Samples

Samples, as used herein, refer to biological samples taken or derived from a subject, e.g., a subject having or suspected of having Celiac disease. Examples of samples include tissue samples or fluid samples. Examples of fluid samples are whole blood, plasma, serum, and other bodily fluids that comprise T cells. In some embodiments, the sample comprises T cells. In some embodiments, the sample comprises T cells and monocytes and/or granulocytes. In some embodiments, the sample comprising T cells comprise whole blood or peripheral blood mononuclear cells (PBMCs). The T cell may be a CD4⁺ T cell, e.g., a gluten-reactive CD4⁺ T cell. In some embodiments, the methods described herein comprise obtaining or providing the sample. In some embodiments, a first and second sample are contemplated. In some embodiments, the first sample is obtained from a subject after administration of a composition comprising a gluten peptide as described herein. In some embodiments, the second sample is obtained after administration of a placebo. Additional samples, e.g., third, fourth, fifth, etc., are also contemplated if additional measurements of a T cell response are desired. Such additional samples may be obtained from the subject at any time, e.g., before or after administration of a composition comprising a gluten peptide and/or a placebo.

### Subjects

A subject may include any subject that is suspected of having Celiac disease. In some embodiments, the subject may include any subject that has or is suspected of having Celiac disease. Preferably, the subject is a human. In some embodiments, the subject has one or more HLA-DQA and HLA-DQB susceptibility alleles encoding HLA-DQ2.5 (DQA1 *05 and DQB1 *02), HLA-DQ2.2 (DQA1 *02 and DQB1 *02) or HLA-DQ8 (DQA1 *03 and DQB1 *0302). In some embodiments, the subject is HLA-DQ2.5 positive (i.e., has both susceptibility alleles DQA1 *05 and DQB1 *02). In some embodiments, a subject may have a family member that has one or more HLA-DQA and HLA-DQB susceptibility alleles encoding HLA-DQ2.5 (DQA1 *05 and DQB1 *02), HLA-DQ2.2 (DQA1 *02 and DQB1 *02) or HLA-DQ8 (DQA1 *03 and DQB1 *0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes.

### Controls and Control Subjects

In some embodiments, methods provided herein comprise measuring a T cell response in a first sample obtained from a subject after administration of a composition comprising a gluten peptide and comparing the T cell response to one or more control T cell responses. In some embodiments, the control T cell response is a T cell response in a sample obtained from the same subject after administration of a placebo.

However, other or further controls are also contemplated. For example, a control T cell response may be a T cell response in a sample from a control subject (or subjects). In some embodiments, a control subject has one or more HLA-DQA and HLA-DQB susceptibility alleles encoding HLA-DQ2.5 (DQA1 *05 and DQB1 *02), DQ2.2 (DQA1 *02 and DQB1 *02) or DQ8 (DQA1 *03 and DQB1 *0302) described herein but does not have Celiac disease. In some embodiments, a control subject does not have any of the HLA-DQA and HLA-DQB susceptibility alleles encoding HLA-DQ2.5 (DQA1 *05 and DQB1 *02), DQ2.2 (DQA1 *02 and DQB1 *02) or DQ8 (DQA1 *03 and DQB1 *0302) described herein. In some embodiments, a control subject is a healthy individual not having or suspected of having Celiac disease. In some embodiments, a control T cell response is a pre-determined value from a control subject or subjects, such that the control T cell response need not be measured every time the methods described herein are performed.

### Gluten Peptides and Compositions Containing Gluten Peptides

As used herein the term “gluten peptide” includes any peptides comprising an amino acid sequence derived from, or encompassed within, one or more of gluten proteins alpha (α), beta (β), γ (γ) and omega (ω) gliadins, and low and high molecular weight (LMW and HMW) glutenins in wheat, B, C and D hordeins in barley, β, γ and CO secalins in rye, and optionally avenins in oats, including deamidated variants thereof containing one or more glutamine to glutamate substitutions. In some embodiments, the composition comprising a gluten peptide in the compositions, kits and methods provided herein is one that comprises one or more gluten proteins. Preferably, in some embodiments, the gluten peptide(s) stimulate a CD4+ T cell specific response.

A gluten peptide may include one or more epitopes known to be recognized by a CD4⁺ T cell in a subject with Celiac disease, e.g., PELP (SEQ ID NO: 12), PELPY (SEQ ID NO: 13), QPELPYP (SEQ ID NO: 89), PQPELPY (SEQ ID NO: 90), FPQPELP, (SEQ ID NO: 91), PELPYPQ (SEQ ID NO: 92), FPQPELPYP (SEQ ID NO: 93), PYPQPELPY (SEQ ID NO:14), PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 5), PQPELPYPQ (SEQ ID NO: 28), FRPEQPYPQ (SEQ ID NO: 29), PQQSFPEQQ (SEQ ID NO: 30), IQPEQPAQL (SEQ ID NO: 31), QQPEQPYPQ (SEQ ID NO: 32), SQPEQEFPQ (SEQ ID NO: 33), PQPEQEFPQ (SEQ ID NO: 34), QQPEQPFPQ (SEQ ID NO: 35), PQPEQPFCQ (SEQ ID NO: 36), QQPFPEQPQ (SEQ ID NO: 37), PFPQPEQPF (SEQ ID NO: 38), PQPEQPFPW (SEQ ID NO: 39), PFSEQEQPV (SEQ ID NO: 40), FSQQQESPF (SEQ ID NO: 41), PFPQPEQPF (SEQ ID NO: 42), PQPEQPFPQ (SEQ ID NO: 43), PIPEQPQPY (SEQ ID NO: 44), PFPQPEQPF (SEQ ID NO: 45), PQPEQPFPQ (SEQ ID NO: 46), PYPEQEEPF (SEQ ID NO: 47), PYPEQEQPF (SEQ ID NO: 48), PFSEQEQPV (SEQ ID NO: 49), EGSFQPSQE (SEQ ID NO: 50), EQPQQPFPQ (SEQ ID NO: 51), EQPQQPYPE (SEQ ID NO: 52), QQGYYPTSPQ (SEQ ID NO: 53), EGSFQPSQE (SEQ ID NO: 54), PQQSFPEQE (SEQ ID NO: 55), or QGYYPTSPQ (SEQ ID NO: 56) (see, e.g., Sollid L M, Qiao S W, Anderson R P, Gianfrani C, Koning F. Nomenclature and listing of celiac disease relevant gluten epitopes recognized by CD4⁺ T cells. Immunogenetics. 2012; 64:455-60; PCT Publication Nos.: WO/2001/025793, WO/2003/104273, WO/2005/105129, and WO/2010/060155). Preferably, in some embodiments, the gluten peptides that comprise epitopes such as those set forth in SEQ ID NO: 12, 13, etc., also comprise additional amino acids flanking either or both sides of the epitope. Exemplary gluten peptides and methods for synthesizing such peptides are known in the art (see, e.g., PCT Publication Nos.: WO/2001/025793, WO/2003/104273, WO/2005/105129, and WO/2010/060155, which are incorporated herein by reference in their entirety). In some embodiments, the gluten peptide comprises PELP (SEQ ID NO: 12), PELPY (SEQ ID NO: 13), QPELPYP (SEQ ID NO: 89), PQPELPY (SEQ ID NO: 90), FPQPELP, (SEQ ID NO: 91), or PELPYPQ (SEQ ID NO: 92) and is at least 8 or 9 amino acids in length.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject (e.g., prior to or during contact of a gluten peptide composition with a sample comprising T cells from a subject) or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 15) could become PFPQPELPY (SEQ ID NO: 1) after processing by tTG. Conservative substitution of E with D is also contemplated herein (e.g., PFPQPELPY (SEQ ID NO: 1) could become PFPQPDLPY (SEQ ID NO: 27). Exemplary peptides including an E to D substitution include peptides comprising or consisting of PFPQPDLPY (SEQ ID NO: 27), PQPDLPYPQ (SEQ ID NO: 94), PFPQPDQPF (SEQ ID NO: 95), PQPDQPFPW (SEQ ID NO: 96), PIPDQPQPY (SEQ ID NO: 97), LQPFPQPDLPYPQPQ (SEQ ID NO: 98), QPFPQPDQPFPWQP (SEQ ID NO: 99), or PQQPIPDQPQPYPQQ (SEQ ID NO: 100). Such substituted peptides can be the gluten peptides of any of the methods and compositions provided herein. Accordingly, gluten peptides that have not undergone deamidation are also contemplated herein (e.g., gluten peptides comprising or consisting of PQLP (SEQ ID NO: 16), PQLPY (SEQ ID NO: 17), QPQLPYP (SEQ ID NO: 101), PQPQLPY (SEQ ID NO: 102), FPQPQLP (SEQ ID NO: 103), PQLPYPQ (SEQ ID NO: 104), FPQPQLPYP (SEQ ID NO: 105), PYPQPQLPY (SEQ ID NO: 18), PFPQPQLPY (SEQ ID NO: 19), PQPQLPYPQ (SEQ ID NO: 20), PFPQPQQPF (SEQ ID NO: 21), PQPQQPFPW (SEQ ID NO: 22), PIPQQPQPY (SEQ ID NO: 23), LQPFPQPQLPYPQPQ (SEQ ID NO: 24), QPFPQPQQPFPWQP (SEQ ID NO: 25), or PEQPIPQQPQPYPQQ (SEQ ID NO: 26), PQPQLPYPQ (SEQ ID NO: 57), FRPQQPYPQ (SEQ ID NO: 58), PQQSFPQQQ (SEQ ID NO: 59), IQPQQPAQL (SEQ ID NO: 60), QQPQQPYPQ (SEQ ID NO: 61), SQPQQQFPQ (SEQ ID NO: 62), PQPQQQFPQ (SEQ ID NO: 63), QQPQQPFPQ (SEQ ID NO: 64), PQPQQPFCQ (SEQ ID NO: 65), QQPFPQQPQ 25 (SEQ ID NO: 66), PFPQPQQPF (SEQ ID NO: 67), PQPQQPFPW (SEQ ID NO: 68), PFSQQQQPV (SEQ ID NO: 69), FSQQQQSPF (SEQ ID NO: 70), PFPQPQQPF (SEQ ID NO: 71), PQPQQPFPQ (SEQ ID NO: 72), PIPQQPQPY (SEQ ID NO: 73), PFPQPQQPF (SEQ ID NO: 74), PQPQQPFPQ (SEQ ID NO: 75), PYPEQQEPF (SEQ ID NO: 76), PYPEQQQPF (SEQ ID NO: 77), PFSQQQQPV (SEQ ID NO: 78), QGSFQPSQQ (SEQ ID NO: 79), QQPQQPFPQ (SEQ ID NO: 80), QQPQQPYPQ (SEQ ID NO: 81), QQGYYPTSPQ (SEQ ID NO: 82), QGSFQPSQQ (SEQ ID NO: 83), PQQSFPQQQ (SEQ ID NO: 84), QGYYPTSPQ (SEQ ID NO: 85), LQPFPQPELPYPQPQ (SEQ ID NO: 86), QPFPQPQQPFPWQP (SEQ ID NO: 87), or PQQPIPQQPQPYPQQ (SEQ ID NO: 88)). In some embodiments, the gluten peptide comprises PQLP (SEQ ID NO: 16), PQLPY (SEQ ID NO: 17), QPQLPYP (SEQ ID NO: 101), PQPQLPY (SEQ ID NO: 102), FPQPQLP (SEQ ID NO: 103), or PQLPYPQ (SEQ ID NO: 104) and is at least 8 or 9 amino acids in length.

A gluten peptide may also be an analog of any of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any of the peptides provided, such variants can include conservative amino acid substitution variants, e.g., E to D substitution.

In some embodiments, analogs may include one or more amino acid substitutions as shown in Table A (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference). The gluten peptides provided herein include analogs of SEQ ID NO:93 comprising one or more of the listed amino acid substitutions. In some embodiments, the analog is an analog of SEQ ID NO: 93 comprising one of the amino acid substitutions provided in Table 1 below. Preferably, analogs generate a T cell response as described herein.

TABLE A
Exemplary substitutions in the epitope FPQPELPYP
(SEQ ID NO: 93)

| Amino acid in | | | | | | | | | |
|---|---|---|---|---|---|---|---|---|---|
| epitope | F | P | Q | P | E | L | P | Y | P |
| Exemplary | A, G, H, I, | A, F, I, M, | A, F, G, | — | D | M | S | I, S, | S, T, |
| Substitutions | L, M P, S, | S, T, V, | H, I, L, | | | | | V, W | Y |
| | T, W, Y | W, Y | M, S, T, | | | | | | |
| | | | V | | | | | | |

The length of the peptide may vary. In some embodiments, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments, peptides are, e.g., 4-1000, 4-500, 4-100, 4-50, 4-40, 4-30, or 4-20 amino acids in length. In some embodiments, peptides are 4-20, 5-20, 6-20, 7-20, 8-20, 9-20, 10-20, 11-20, 12-20, 13-20, 14-20, or 15-20 amino acids in length. In some embodiments, peptides are e.g., 5-30, 10-30, 15-30 or 20-30 amino acids in length. In some embodiments, peptides are 4-50, 5-50, 6-50, 7-50, 8-50, 9-50, 10-50, 11-50, 12-50, 13-50, 14-50, or 15-50 amino acids in length. In some embodiments, peptides are 8-50 amino acids in length.

In some embodiments, a composition comprising at least one or one or more gluten peptide(s) is contemplated. In some embodiments, the methods described herein comprise administering the composition to a subject (e.g., a subject having or suspected of having Celiac disease). In some embodiments, the methods described herein comprise contacting the composition with a sample from a subject (e.g., a sample comprising T cells).

In some embodiments, the composition comprises at least one of a wheat gluten, a barley hordein, and a rye secalin. In some embodiments, the composition comprises at least two of a wheat gluten, a barley hordein, or a rye secalin. In some embodiments, the composition comprises a wheat gluten, a barley hordein, and a rye secalin. In some embodiments, the composition comprises a consistently known amount of a wheat gluten, a barley hordein, and/or a rye secalin. For example, the amount of wheat gluten, barley hordein, and/or rye secalin may be standardized such that each composition for each subject contains the same amount of wheat gluten, barley hordein, and/or rye secalin. In some embodiments, the wheat gluten, barley hordein, and/or rye secalin are present in an amount of at least 500 mg, e.g., 500 mg to 10 grams. Multiple compositions of wheat gluten, barley hordein, and/or rye secalin may be administered to a subject such that the subject receives at least 1 gram per day of wheat gluten, barley hordein, and/or rye secalin, e.g., 1 to 10 grams per day of wheat gluten, barley hordein, and/or rye secalin. Preferably, such compositions test positive with an R5-ELISA.

In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). “First”, “second”, and “third” are not meant to imply an order of use or importance, unless specifically stated otherwise. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 6); the second peptide comprises the amino acid sequence QPFPQPEQPFPWQP (SEQ ID NO: 7); and/or the third peptide comprises the amino acid sequence PEQPIPEQPQPYPQQ (SEQ ID NO: 8).

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, in some embodiments, the composition comprises at least one of: (i) a first peptide comprising the amino acid sequence PFPQPQLPY (SEQ ID NO: 19) and PQPQLPYPQ (SEQ ID NO: 20), (ii) a second peptide comprising the amino acid sequence PFPQPQQPF (SEQ ID NO: 21) and PQPQQPFPW (SEQ ID NO: 22), and (iii) a third peptide comprising the amino acid sequence PIPQQPQPY (SEQ ID NO: 23). In some embodiments, the first peptide comprises the amino acid sequence LQPFPQPQLPYPQPQ (SEQ ID NO: 86); the second peptide comprises the amino acid sequence QPFPQPQQPFPWQP (SEQ ID NO: 87); and/or the third peptide comprises the amino acid sequence PQQPIPQQPQPYPQQ (SEQ ID NO: 88).

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

Particular changes can be made to a gluten peptide to improve resistance to degradation or optimize solubility properties or otherwise improve bioavailability compared to the parent gluten peptide, thereby providing gluten peptides having similar or improved therapeutic, diagnostic and/or pharmacokinetic properties. A preferred such modification, in some embodiments, includes the use of an N-terminal acetyl group or pyroglutamate and/or a C-terminal amide. Such modifications have been shown in the art to significantly increase the half-life and bioavailability of peptides compared to the peptides having a free N- and C-terminus (see, e.g., PCT Publication No.: WO/2010/060155). In some embodiments, the first, second and/or third peptides comprise an N-terminal acetyl group or pyroglutamate group and/or a C-terminal amide group. In some embodiments, the first peptide comprises the amino acid sequence ELQPFPQPELPYPQPQ (SEQ ID NO: 9), wherein the N-terminal E is a pyroglutamate; the second peptide comprises the amino acid sequence EQPFPQPEQPFPWQP (SEQ ID NO: 10), wherein the N-terminal E is a pyroglutamate; and/or the third peptide comprises the amino acid sequence EPEQPIPEQPQPYPQQ (SEQ ID NO: 11), wherein the N-terminal E is a pyroglutamate. In some embodiments, the first peptide consists of the amino acid sequence ELQPFPQPELPYPQPQ (SEQ ID NO: 9), wherein the N-terminal E is a pyroglutamate; the second peptide consists of the amino acid sequence EQPFPQPEQPFPWQP (SEQ ID NO: 10), wherein the N-terminal E is a pyroglutamate; and/or the third peptide consists of the amino acid sequence EPEQPIPEQPQPYPQQ (SEQ ID NO: 11), wherein the N-terminal E is a pyroglutamate. In some embodiments, the first peptide consists of the amino acid sequence ELQPFPQPELPYPQPQ (SEQ ID NO: 9), wherein the N-terminal E is a pyroglutamate, and wherein the peptide contains a C-terminal amide group; the second peptide consists of the amino acid sequence EQPFPQPEQPFPWQP (SEQ ID NO: 10), wherein the N-terminal E is a pyroglutamate, and wherein the peptide contains a C-terminal amide group; and/or the third peptide consists of the amino acid sequence EPEQPIPEQPQPYPQQ (SEQ ID NO: 11), wherein the N-terminal E is a pyroglutamate, and wherein the peptide contains a C-terminal amide group.

### Other Testing

In some embodiments, methods described herein comprise other testing of a subject (e.g., based on the results of the methods described herein). As used herein, “other testing” describes use of at least one additional diagnostic method in addition to the methods provided herein. Any diagnostic method or combination thereof for Celiac disease is contemplated as other testing. Exemplary other testing includes, but is not limited to, intestinal biopsy, serology (measuring the levels of one or more antibodies present in the serum), and genotyping (see, e.g., Walker-Smith J A, et al. Arch Dis Child 1990). Such other testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles).

When performing intestinal biopsies, generally multiple biopsies are taken from the second or third part of the duodenum. Endoscopy has become the most convenient method of obtaining biopsies of the small-intestinal mucosa. Suction biopsy (with a Crosby capsule) can provide the best samples. Celiac disease (CD) affects the mucosa of the proximal small intestine, with damage gradually decreasing in severity towards the distal small intestine, although in severe cases the lesions can extend to the ileum. The degree of proximal damage varies greatly depending on the severity of the disease. The proximal damage may be very mild in “silent” cases, with little or no abnormality detectable histologically in the mid-jejunum. Abnormalities in the gastric and rectal mucosa may be observed in some cases. Occasionally, the lesion in the duodenum/upper jejunum can be patchy, which may justify a second biopsy immediately in selected patients with positive endomysial antibody (EMA). However, this is only warranted if all three samples of the first biopsy show a normal histology.

Detection of serum antibodies (serology) is also contemplated. The presence of such serum antibodies can be detected using methods known to those of skill in the art, e.g., by ELISA, histology, cytology, immunofluorescence or western blotting. Such antibodies include, but are not limited to: IgA ant-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1 *05 and DQB1 *02), DQ2.2 (DQA1 *02 and DQB1 *02) or DQ8 (DQA1 *03 and DQB1 *0302). Exemplary sequences that encode the DQA and DQB susceptibility alleles include HLA-DQA1 *0501 (Genbank accession number: AF515813.1) HLA-DQA1 *0505 (AH013295.2), HLA-DQB1 *0201 (AY375842.1) or HLA-DQB1 *0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### Treatment

In some embodiments, the methods described herein comprise a treatment step, such as treating a subject identified as having Celiac disease. In some embodiments, the methods can comprise a step where information regarding treatment is provided to the subject. Such information can be given orally or in written form, such as with written materials. Written materials may be in an electronic form. Any known treatment of Celiac disease is contemplated herein. Exemplary treatments include, e.g., a gluten-free diet. Other exemplary treatments include endopeptidases, such as ALV003 (Alvine) and AT1001 (Alba), agents that inhibit transglutaminase activity, agents that block peptide presentation by HLA DQ2.5, or oral resins that bind to gluten peptides and reduce their bioavailability.

Compositions comprising gluten peptides for use in treating Celiac disease are known in the art (see, e.g., PCT Publication Nos.: WO/2001/025793, WO/2003/104273, WO/2005/105129, and WO/2010/060155, which are incorporated herein by reference in their entirety). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 6); the second peptide comprises the amino acid sequence QPFPQPEQPFPWQP (SEQ ID NO: 7); and/or the third peptide comprises the amino acid sequence PEQPIPEQPQPYPQQ (SEQ ID NO: 8). Modifications to such peptides, e.g., an N-terminal pyro-glutamate and/or C-terminal amide, are contemplated and described herein. In some embodiments, the first peptide consists of the amino acid sequence ELQPFPQPELPYPQPQ (SEQ ID NO: 9), wherein the N-terminal E is a pyroglutamate, and wherein the first peptide contains a C-terminal amide group; the second peptide consists of the amino acid sequence EQPFPQPEQPFPWQP (SEQ ID NO: 10), wherein the N-terminal E is a pyroglutamate, and wherein the second peptide contains a C-terminal amide group; and/or the third peptide consists of the amino acid sequence EPEQPIPEQPQPYPQQ (SEQ ID NO: 11), wherein the N-terminal E is a pyroglutamate, and wherein the third peptide contains a C-terminal amide group.

Treatments may be administrated through any method known in the art. Pharmaceutical compositions suitable for each administration route are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005).

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. Pharmaceutical compositions may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical compositions include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005).

It is especially advantageous to formulate the active agent in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington's Pharmaceutical Sciences, 16th Ed. Mack Publishing Company, 1980 and Remington: The Science and Practice of Pharmacy, 21st Ed. Lippincott Williams & Wilkins, 2005. Effective amounts may be measured from ng/kg body weight to g/kg body weight per minute, hour, day, week or month.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Kits

Another aspect of the disclosure relates to kits. In some embodiments, the kit comprises a composition comprising one or more of any of the gluten peptide compositions as described herein and a placebo as described herein. In some embodiments, the composition and the placebo are foodstuffs (e.g., baked goods such as breads, cookies, muffins, cakes, etc.). In some embodiments, the composition comprises at least one of a wheat gluten, a barley hordein, and a rye secalin. In some embodiments, the composition comprises at least two of a wheat gluten, a barley hordein, and a rye secalin. In some embodiments, the composition comprises a wheat gluten, a barley hordein, and a rye secalin. In some embodiments, the composition and placebo are each labeled with a generic label (e.g., “A” and “B”) such that the two may be distinguished from one another but not so that a subject reading the generic label can conclude which is the gluten peptide composition and which is the placebo.

In some embodiments, the kit comprises or further comprises: (a) a composition comprising at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5); and/or (b) an agent for assessing a T cell response. In some embodiments, the agent is a binding partner for a cytokine indicative of the T cell response. In some embodiments, the kit further comprises an agent that recognizes the binding partner for, for example, IFN-γ.

In some embodiments, the composition contained in the kit comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and/or the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and/or the third peptide comprises EPEQPIPEQPQPYPQQ (SEQ ID NO: 11), wherein the N-terminal E is a pyroglutamate. In some embodiments, the first peptide consists of ELQPFPQPELPYPQPQ (SEQ ID NO: 9), wherein the N-terminal E is a pyroglutamate; the second peptide consists of EQPFPQPEQPFPWQP (SEQ ID NO: 10), wherein the N-terminal E is a pyroglutamate; and/or the third peptide consists of EPEQPIPEQPQPYPQQ (SEQ ID NO: 11), wherein the N-terminal E is a pyroglutamate. In some embodiments, the first peptide consists of ELQPFPQPELPYPQPQ (SEQ ID NO: 9), wherein the N-terminal E is a pyroglutamate, and wherein the peptide contains a C-terminal amide group; the second peptide consists of EQPFPQPEQPFPWQP (SEQ ID NO: 10), wherein the N-terminal E is a pyroglutamate, and wherein the peptide contains a C-terminal amide group; and/or the third peptide consists of EPEQPIPEQPQPYPQQ (SEQ ID NO: 11), wherein the N-terminal E is a pyroglutamate, and wherein the peptide contains a C-terminal amide group.

In some embodiments, the kit further comprises a container for whole blood. In some embodiments, the gluten peptide composition is contained within the container (e.g., dried onto the wall of the container). In some embodiments, the composition is contained within a solution separate from the container, such that the composition may be added to the container after blood collection. In some embodiments, the composition is in lyophilized form in a separate container, such that the composition may be reconstituted and added to the container after blood collection, in some embodiments. In some embodiments, the container further contains an anti-coagulant, such as heparin. In some embodiments, the container is structured to hold a defined volume of blood e.g. 1 mL or 5 mL. In some embodiments, the container is present in the kit in duplicate or triplicate.

In some embodiments, the kit further comprises a negative control container for whole blood and/or a positive control container for whole blood. The negative control container may be, for example, an empty container or a container containing a non-T cell-activating peptide (e.g., dried onto the wall of the container), such as a non-CD4⁺-T cell-activating peptide. The positive control container may contain, for example, a mitogen such as PHA-L (e.g., 10 units PHA-L). In some embodiments, the negative control container and/or positive control container are structured to hold a defined volume of blood e.g. 1 mL or 5 mL. In some embodiments, the negative control container and/or positive control container are present in the kit in duplicate or triplicate. In some embodiments, the kit comprises any combination of the components mentioned above.

Any suitable binding partner is contemplated. In some embodiments, the binding partner is any molecule that binds specifically to a cytokine as provided herein. As described herein, “binds specifically” means that the molecule is more likely to bind to a portion of or the entirety of a protein to be measured than to a portion of or the entirety of another protein. In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is any molecule that binds specifically to an IFN-γ mRNA. As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments, the binding partner that binds specifically to an mRNA is a nucleic acid, e.g., a probe.

In some embodiments, the kit further comprises a first and second binding partner for a cytokine provided herein. In some embodiments, the first and second binding partners are antibodies or antigen binding fragments thereof. In some embodiments, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner is washed over the cytokine bound to the second binding partner (e.g., as in a sandwich ELISA). The first binding partner may comprise a detectable label, or an agent that recognizes the first binding partner (e.g., a secondary antibody) may comprise a detectable label.

Any suitable agent that recognizes a binding partner is contemplated. In some embodiments, the binding partner is any molecule that binds specifically to the binding partner. In some embodiments, the agent is an antibody (e.g., a secondary antibody) or antigen-binding fragment thereof, such as Fab, F(ab)2, Fv, single chain antibodies, Fab and sFab fragments, F(ab′)2, Fd fragments, scFv, or dAb fragments. Agents also include other peptide molecules and aptamers that bind specifically to a binding partner. In some embodiments, the binding partner comprises a biotin moiety and the agent is a composition that binds to the biotin moiety (e.g., an avidin or streptavidin).

In some embodiments, the binding partner and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art and can be detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments, the kit further comprises instructions for performing a challenge method provided herein and/or for detecting a T cell response (e.g., detecting a cytokine indicative of the T cell response) in a sample from a subject having or suspected of having Celiac disease. In some embodiments, the instructions include the methods described herein. Instructions can be in any suitable form, e.g., as a printed insert or a label.

### EXAMPLES

### Example 1

### Methods

Gluten-containing and gluten-free cookies were prepared that shared similar taste, texture and appearance. Volunteers were subjects with Celiac disease diagnosed according to standard criteria (Walker-Smith J A, et al. Arch Dis Child 1990), and following a strict gluten free diet. All subjects were confirmed to have the HLA DQA1 *05 and HLA DQB1 *02 alleles encoding HLA DQ2.5. On days 1-3, and days 8-10, volunteers consumed three cookies daily that either contained gluten or were a placebo that did not contain gluten confirmed by R5 ELISA (Dairy Testing Services Food Laboratories, Kensington, VIC, Australia and Mendez E, et al. Eur J Gastroenterol Hepatol. 2005.). One milliliter of whole blood was collected in the morning on Days 0, 1, 6, 8, 13, and 21 directly into three Quantiferon Gold NIL tubes (containing no antigen, T0591-0205; Cellestis International, Clayton VIC Australia) and one MITOGEN tube (T0593-0201). Sterile phosphate buffered saline (PBS) was added to whole blood in one NIL tube and the MITOGEN tube by injecting 0.1 mL through the cap using sterile 0.5 mL Terumo syringes fitted with 29 G needles. A gluten peptide mixture containing the following three peptides:

| | | |
|---|---|---|
| (a) | ELQPFPQPELPYPQPQ, | (SEQ ID NO: 9) |
| (b) | EQPFPQPEQPFPWQP, | (SEQ ID NO: 10) |
| and | | |
| (c) | EPEQPIPEQPQPYPQQ, | (SEQ ID NO: 11) |

wherein the N-terminal E in each peptide is a pyroglutamate and the C-terminus of each peptide contains an amide group, and wherein each peptide is present in an amount of approximately 9 mg/mL, was diluted in PBS to 1.5 mg/mL and 0.1 mL added to one NIL tube containing 1 mL whole blood. 0.1 mL of a pool of 23 peptides corresponding to MHC class I-restricted T cell epitopes from human cytomegalovirus, Epstein Barr virus and influenza virus, CEF (CEF peptide pool, 3615-1; MABTECH AB, SE-131 28 Nacka Strand Sweden) diluted in PBS to a final concentration of 10 ug/mL was added to the third NIL tube. Tubes were inverted ten times and then incubated for 24 h in a 37° C. 5% CO2 incubator. Blood tubes were centrifuged 2000 g for 15 min, and plasma aspirated and frozen at −20° C. When desired, the plasma was thawed for interferon-γ (IFN-γ) measurement by ELISA (ELISA for Human IFN-γ, Product Code: 3420-1H-20; MABTECH AB, SE-131 28 Nacka Strand Sweden). Samples were measured in triplicate and considered to be “positive” if the mean of three interferon-γ level readings to the gluten peptide mixture after subtraction of the response to NIL was greater than 7.2 pg/mL and the ratio of interferon-γ levels in the gluten peptide mixture tube to NIL tube was over 1.25. Responses were considered negative if interferon-γ levels were below 7.2 pg/mL after subtraction of the response to NIL and/or the ratio of interferon-γ levels in the gluten peptide mixture tube to NIL tube was over 1.25. Individual patient responses to gluten-placebo challenge were considered overall “positive” if gluten was ingested on Days 1-3 and interferon-γ was positive on Day 6, or if gluten was ingested on Days 8-10 then interferon-γ was negative on Day 6 and positive on Day 13. Individual patient responses to gluten-placebo challenge were considered overall “negative” if placebo was ingested on Days 1-3 and interferon-γ was positive on Day 6.

TABLE 2
Participant details and baseline serology (Normal < 20 U)

| | | | Baseline Serology (Normal < 20 U) | | |
|---|---|---|---|---|---|
| Subject | Age | Sex | tTG | DGP-IgG | DGP-IgA |
| 1 | 61 | F | 2.5 | 4 | 2 |
| 2 | 64 | F | 10.5 | 4 | 4 |
| 3 | 69 | F | 2.1 | 3 | 4 |
| 4 | 35 | M | 20.0 | 6 | 12 |
| 5 | 52 | M | 12.2 | 2 | 7 |
| 6 | 56 | F | 6.9 | 8 | 8 |
| 7 | 49 | F | 9.8 | 8 | 21 |
| 8 | 30 | F | 5.8 | 3 | 1 |
| 9 | 41 | M | 7.3 | 6 | 3 |
| 10 | 36 | F | 3.7 | 4 | 3 |
| 11 | 68 | F | 3.3 | 3 | 12 |
| 12 | 59 | M | 1.6 | 6 | 3 |
| 13 | 51 | F | 10.8 | 6 | 4 |
| 14 | 61 | F | 5.4 | 4 | 5 |
| 15 | 69 | M | 1.3 | 23 | 6 |

### Results

Fifteen HLA DQ2.5⁺ subjects with Celiac disease following a strict gluten free diet were enrolled and completed the study protocol (Tables 2 and 3). Technical issues with the ELISA assay in 9 subjects led to exclusion of their data from further analysis. Celiac disease specific serology was negative or borderline positive consistent with subjects following gluten free diet. Each subject was given cookies (either gluten-containing or placebo) to consume 3 times a day for 3 days (days 1-3 of the study). On days 8-10 of the study, the subjects consumed the opposite type of cookie from the type consumed during days 1-3. Whole blood interferon-γ release to MITOGEN and CEF were greater than NIL in each assay for all subjects. According to the criteria set out for gluten peptide mixture whole blood interferon-γ release responses to gluten and placebo challenges in the methods section above, six subjects showed “positive” responses (Table 4) and none were negative.

TABLE 3
Cookies consumed per subject

| | Cookie Set | | | |
|---|---|---|---|---|
| Subject | 1 | Code | Cookie Set 2 | Code |
| 1 | 3/3/3 | 1ABC | 3/3/3 | 8ABC |
| 2 | 3/3/3 | 2ABC | 3/3/3 | 9ABC |
| 3 | 2/3/3* | 3ABC | 3/3/3 | 6ABC |
| 4 | 3/3/3 | 4ABC | 3/3/3 | 10ABC |
| 5 | 3/3/3 | 5ABC | 1/0/0* | 7ABC |
| 6 | 3/3/3 | 11ABC | 3/3/3 | 11DEF |
| 7 | 3/3/3 | 12ABC | 1/0/0* | 12DEF |
| 8 | 3/3/3 | 13ABC | 2/3/3* | 13DEF |
| 9 | 2/3/3* | 14ABC | 3/3/3 | 14DEF |
| 10 | 3/3/3 | 15ABC | 3/3/3 | 15DEF |
| 11 | 3/3/3 | 16ABC | 3/3/3 | 16DEF |
| 12 | 3/3/3 | 19ABC | 3/3/3 | 19DEF |
| 13 | 3/3/3 | 18ABC | 3/3/3 | 18DEF |
| 14 | 3/3/3 | 17ABC | 3/3/3 | 17DEF |
| 15 | 3/3/3 | 20ABC | 3/3/3 | 20DEF |

* = incomplete 3-day cookie challenge

TABLE 4
Gluten peptide mixture-specific interferon-γ responses to blinded
placebo-controlled oral gluten challenge

| | | Day 6 | | Day 13 |
|---|---|---|---|---|
| | | Gluten peptide | | Gluten peptide |
| | | mixture whole | | mixture whole |
| | Challenge | blood IFNg | Challenge Days | blood IFNg |
| Subject | Days 1-3 | release assay | 8-10 | release assay |
| 1 | Gluten | Positive | Placebo | Positive |
| 3 | Gluten | Positive | Placebo | Negative |
| 5 | Placebo | Negative | Gluten | Positive |
| 8 | Placebo | Negative | Gluten | Positive |
| 10 | Placebo | Negative | Gluten | Positive |
| 12 | Gluten | Positive | Placebo | Negative |

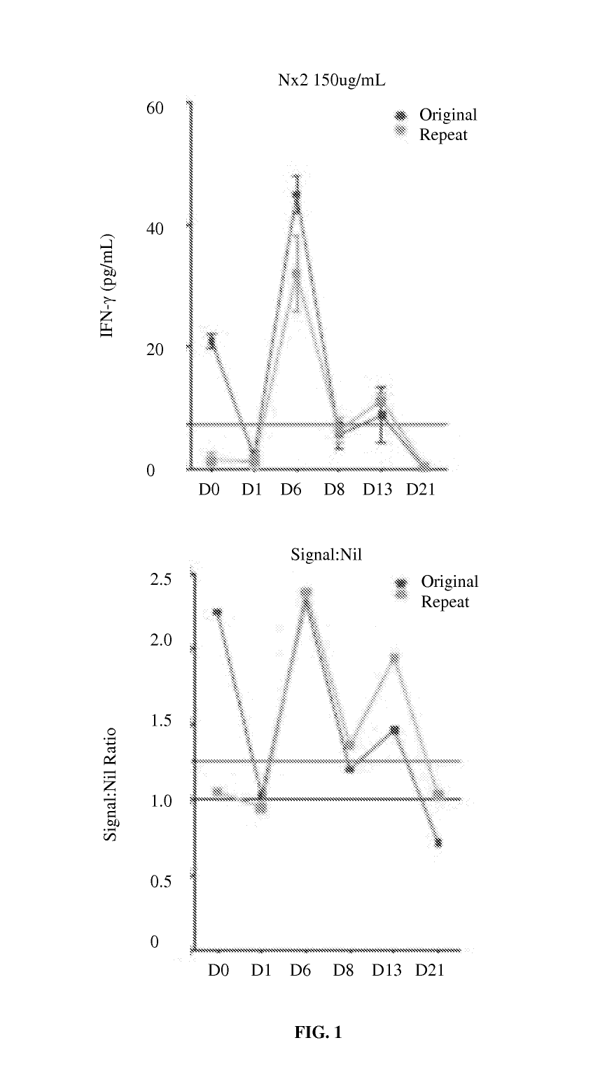

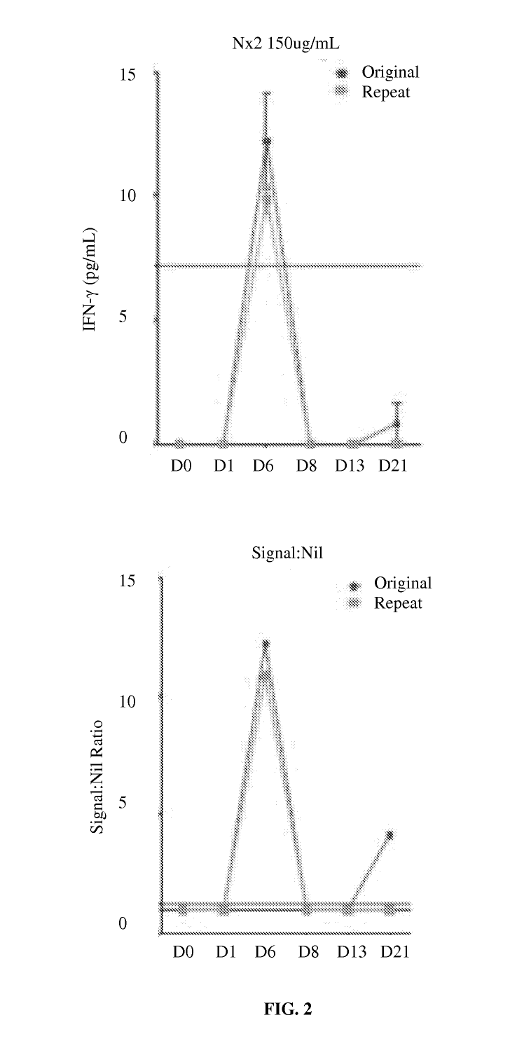

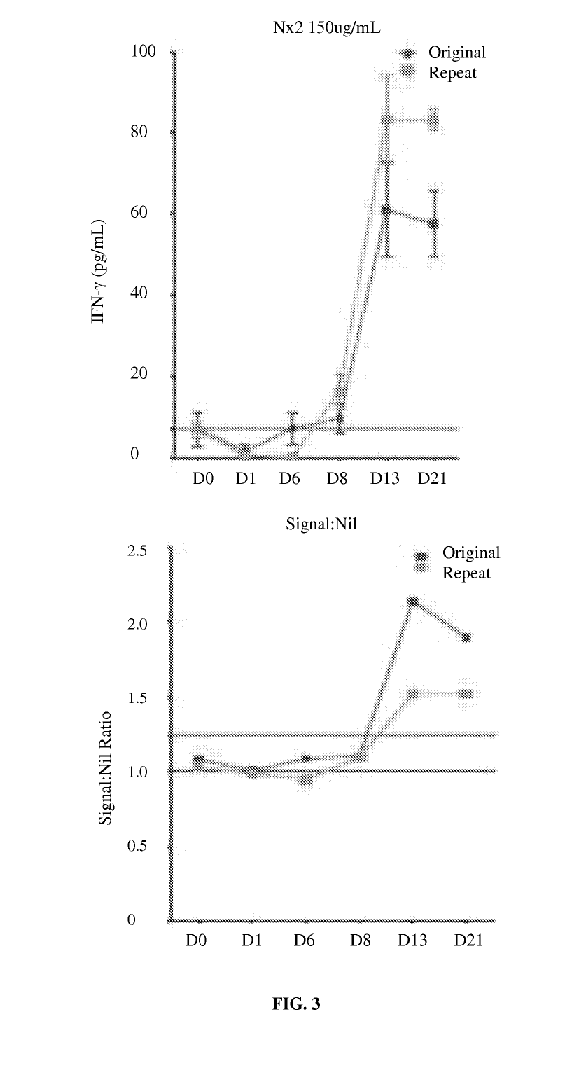

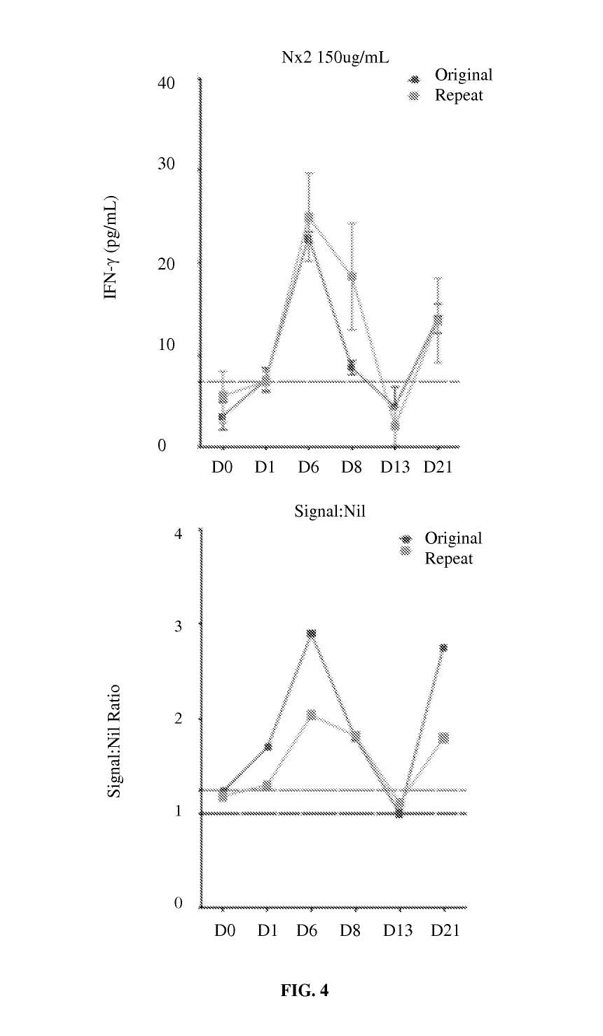

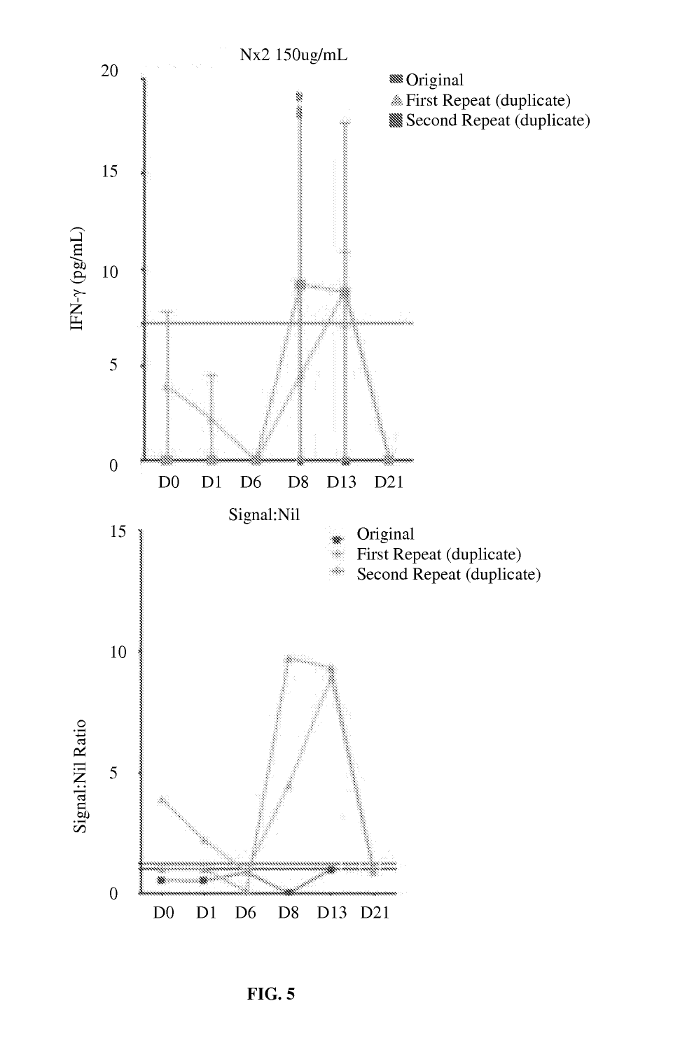

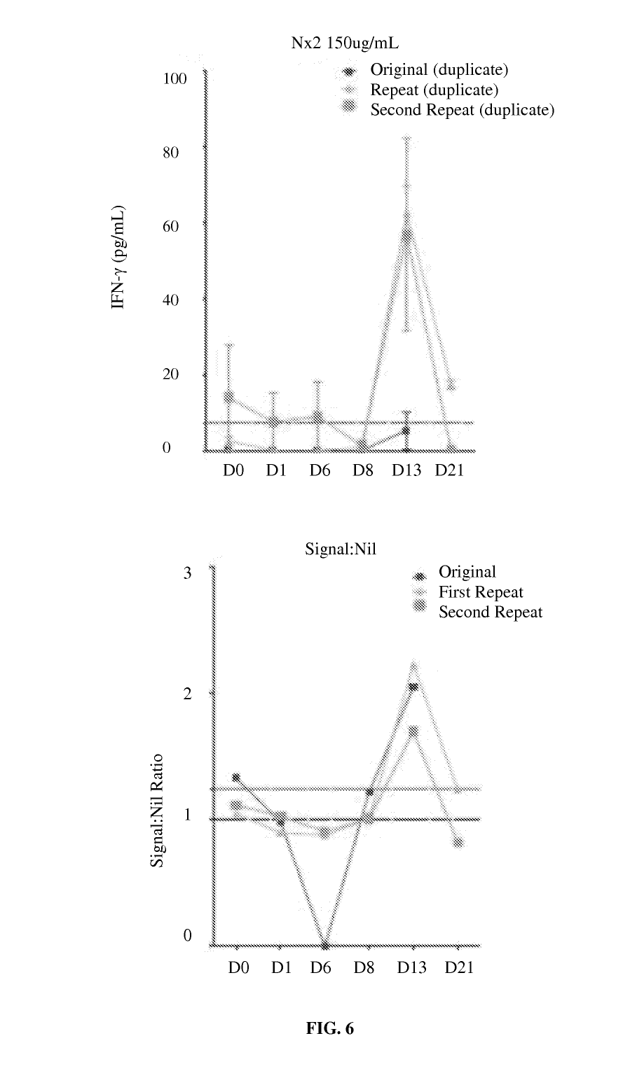

Exemplary positive and negative responses in subjects on day 6 and day 13 are shown in FIGS. 1-6. FIGS. 1-6 show that, in general, IFN-γ levels rose by 3 days after consumption of the gluten-containing cookies (either on day 6 or 13, depending on when the subject consumed the gluten-containing cookies). This is summarized and further supported in Table 4, which show that, with the exception of one subject, 5 subjects with Celiac disease had a positive IFN-γ response 3 days after challenge (>7.5 pg/mL by IFN-γ ELISA assay) when given the gluten-containing cookies and a negative IFN-γ response 3 days after challenge (<7.5 pg/mL by IFN-γ ELISA assay) when given the placebo non-gluten-containing cookies. Overall, the data presented herein show that a placebo-controlled oral challenge can identify a subject as having Celiac disease while minimizing the time that the subject exposes themselves to gluten, resulting in a more tolerable test.

### REFERENCES

1. Baker, P. G. & Read, A. E. Oats and barley toxicity in coeliac disease. Postgrad Med J 1976, 52: 264-268.
2. Dicke W K. Coeliac Disease. Investigation of the Harmful Effects of Certain Types of Cereal on Patients with Coeliac Disease (Thesis). The Netherlands: University of Utrecht, 1950.
3. Dicke W K, Weijers H A, Van De Kamer J H. Coeliac disease. II. The presence in wheat of a factor having a deleterious effect in cases of coeliac disease. Acta Paediatr. 1953; 42(1): 34-42.
4. Hadithi M, von Blomberg B M, Crusius J B, Bloemena E, Kostense P J, Meijer J W, Mulder C J, Stehouwer C D, Peña A S. Accuracy of serologic tests and HLA-DQ typing for diagnosing Celiac disease. Ann Intern Med. 2007 Sep. 4; 147(5):294-302.
5. Husby S, Koletzko S, Korponay-Szabó I R, Mearin M L, Phillips A, Shamir R, Troncone R, Giersiepen K, Branski D, Catassi C, Lelgeman M, Mäki M, Ribes-Koninckx C, Ventura A, Zimmer K P; ESPGHAN Working Group on Coeliac Disease Diagnosis; ESPGHAN Gastroenterology Committee; European Society for Pediatric Gastroenterology, Hepatology, and Nutrition. European Society for Pediatric Gastroenterology, Hepatology, and Nutrition guidelines for the diagnosis of coeliac disease. J Pediatr Gastroenterol Nutr. 2012 January; 54(1):136-60.
6. Karell K, Louka A S, Moodie S J, Ascher H, Clot F, Greco L, Ciclitira P J, Sollid L M, Partanen J; European Genetics Cluster on Celiac Disease. HLA types in Celiac disease patients not carrying the DQA1 *05-DQB1 *02 (DQ2) heterodimer: results from the European Genetics Cluster on Celiac Disease. Hum Immunol. 2003 April; 64(4):469-77.
7. Koskinen L, Romanos J, Kaukinen K, Mustalahti K, Korponay-Szabo I, Barisani D, Bardella M T, Ziberna F, Vatta S, Széles G, Pocsai Z, Karell K, Haimila K, Adány R, Not T, Ventura A, Mäki M, Partanen J, Wijmenga C, Saavalainen P. Cost-effective HLA typing with tagging SNPs predicts Celiac disease risk haplotypes in the Finnish, Hungarian, and Italian populations. Immunogenetics. 2009 April; 61(4):247-56. Epub 2009 Mar. 3.
8. Kumar P J, O'Donoghue D P, Stenson K, Dawson A M. Reintroduction of gluten in adults and children with treated coeliac disease. Gut. 1979 September; 20(9):743-9.
9. Mendez E, Vela C, Immer U, Janssen F W. Report of a collaborative trial to investigate the performance of the R5 enzyme linked immunoassay to determine gliadin in gluten-free food. Eur J Gastroenterol Hepatol. 2005 October; 17(10):1053-63.
10. Van De Kamer J H, Weyers H A, Dicke W K. Coeliac disease IV. An investigation into the injurious constituents of wheat in connection with their action on patients with coeliac disease. Acta Paediatr. 1953; 42: 223-231.
11. Walker-Smith J A, et al. Revised criteria for diagnosis of coeliac disease. Report of Working Group of European Society of Paediatric Gastroenterology and Nutrition. Arch Dis Child 1990; 65:909-11.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of assessing a T cell response in a subject, the method comprising:
(a) measuring a T cell response in a first sample comprising T cells obtained from the subject after administration to the subject of a first composition comprising at least one gluten peptide; and
(b) measuring a T cell response in a second sample comprising T cells obtained from the subject after administration of a placebo.

2. A method of assessing a T cell response in a subject, the method comprising:
(a) administering to the subject a first composition, the first composition comprising at least one gluten peptide;
(b) measuring a T cell response in a first sample comprising T cells obtained from the subject after administration of the first composition;
(c) administering to the subject a placebo; and
(d) measuring a T cell response in a second sample comprising T cells obtained from the subject after administration of the placebo.

3. The method of claim 1 or 2, wherein the first composition is administered to the subject before the placebo is administered to the subject.

4. The method of claim 1 or 2, wherein the placebo is administered to the subject before the first composition is administered to the subject.

5. The method of any one of claims 1 to 4, wherein the measuring of a T cell response in the first sample and the second sample are performed together in one assay.

6. The method of any one of claims 1 to 5, wherein the sample comprises whole blood or peripheral blood mononuclear cells.

7. The method of any one of claims 1 to 6, wherein the first composition is administered to the subject more than once.

8. The method of claim 7, wherein the first composition is administered to the subject at least once a day for three days.

9. The method of claim 8, wherein the first composition is administered to the subject three times a day for three days.

10. The method of any one of claims 1 to 9, wherein the placebo is administered to the subject more than once.

11. The method of claim 10, wherein the placebo is administered to the subject at least once a day for three days.

12. The method of claim 11, wherein the placebo is administered to the subject three times a day for three days.

13. The method of any one of claims 1 to 12, wherein the administration of the first composition and the placebo is oral administration.

14. The method of claim 13, wherein the first composition and the placebo are foodstuffs.

15. The method of any one of claims 1 to 14, wherein the measuring of a T cell response in the first sample and the second sample comprises contacting the first and second samples with a second composition comprising at least one gluten peptide and measuring the level of at least one cytokine in the first and second samples.

16. The method of claim 15, wherein the second composition contacted with the first sample and the second sample is the same composition.

17. The method of claim 15 or 16, wherein the at least one cytokine is IFN-γ.

18. The method of claim 17, wherein the method further comprises comparing the T cell response measured in the first sample with a control T cell response and comparing the T cell response measured in the second sample to the control T cell response.

19. The method of any one of claims 15 to 18, wherein the level of the at least one cytokine is measured with an enzyme-linked immunosorbent assay (ELISA).

20. The method of any one of claims 15 to 18, wherein the level of the at least one cytokine is measured with an enzyme-linked immunosorbent spot (ELISpot) assay.

21. The method of any one of claims 1 to 20, wherein the first composition comprises at least one of:
(i) a first peptide comprising the amino acid sequence PFPQPELPY (SEQ ID NO: 1), or a non-deamidated version thereof, and PQPELPYPQ (SEQ ID NO: 2), or a non-deamidated version thereof,
(ii) a second peptide comprising the amino acid sequence PFPQPEQPF (SEQ ID NO: 3), or a non-deamidated version thereof, and PQPEQPFPW (SEQ ID NO: 4), or a non-deamidated version thereof, or
(iii) a third peptide comprising the amino acid sequence PIPEQPQPY (SEQ ID NO: 5), or a non-deamidated version thereof.

22. The method of any one of claims 15-21, wherein the second composition comprises at least one of:
(i) a first peptide comprising the amino acid sequence PFPQPELPY (SEQ ID NO: 1), or a non-deamidated version thereof, and PQPELPYPQ (SEQ ID NO: 2), or a non-deamidated version thereof,
(ii) a second peptide comprising the amino acid sequence PFPQPEQPF (SEQ ID NO: 3), or a non-deamidated version thereof, and PQPEQPFPW (SEQ ID NO: 4), or a non-deamidated version thereof, or
(iii) a third peptide comprising the amino acid sequence PIPEQPQPY (SEQ ID NO: 5), or a non-deamidated version thereof.

23. The method of claim 21 or 22, wherein the first peptide comprises LQPFPQPQLPYPQPQ (SEQ ID NO: 86); the second peptide comprises QPFPQPQQPFPWQP (SEQ ID NO: 87); and/or the third peptide comprises PQQPIPQQPQPYPQQ (SEQ ID NO: 88).

24. The method of any one of claims 15-21, wherein the second composition comprises at least one of:
(i) a first peptide comprising the amino acid sequence PFPQPDLPY (SEQ ID NO: 27) and PQPDLPYPQ (SEQ ID NO: 94),
(ii) a second peptide comprising the amino acid sequence PFPQPDQPF (SEQ ID NO: 95) and PQPDQPFPW (SEQ ID NO: 96), or
(iii) a third peptide comprising the amino acid sequence PIPDQPQPY (SEQ ID NO: 97).

25. The method of claim 24, wherein the first peptide comprises LQPFPQPDLPYPQPQ (SEQ ID NO: 98), the second peptide comprises QPFPQPDQPFPWQP (SEQ ID NO: 99), and/or the third peptide comprises PQQPIPDQPQPYPQQ (SEQ ID NO: 100).

26. The method of claim 21, wherein the first peptide comprises LQPFPQPELPYPQPQ (SEQ ID NO: 6); the second peptide comprises QPFPQPEQPFPWQP (SEQ ID NO: 7); and/or the third peptide comprises PEQPIPEQPQPYPQQ (SEQ ID NO: 8).

27. The method of any one of claims 21 to 26, wherein the first, second and/or third peptides comprise an N-terminal acetyl group or pyroglutamate group, and/or a C terminal amide group.

28. The method of claim 22, wherein the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

29. The method of claim 27, wherein the first peptide consists of ELQPFPQPELPYPQPQ (SEQ ID NO: 9), wherein the N-terminal E is a pyroglutamate; the second peptide consists of EQPFPQPEQPFPWQP (SEQ ID NO: 10), wherein the N-terminal E is a pyroglutamate; and the third peptide consists of EPEQPIPEQPQPYPQQ (SEQ ID NO: 11), wherein the N-terminal E is a pyroglutamate.

30. The method of claim 27, wherein the first peptide consists of ELQPFPQPELPYPQPQ (SEQ ID NO: 9), wherein the N-terminal E is a pyroglutamate, and wherein the first peptide contains a C-terminal amide group; the second peptide consists of EQPFPQPEQPFPWQP (SEQ ID NO: 10), wherein the N-terminal E is a pyroglutamate, and wherein the second peptide contains a C-terminal amide group; and/or the third peptide consists of EPEQPIPEQPQPYPQQ (SEQ ID NO: 11), wherein the N-terminal E is a pyroglutamate, and wherein the third peptide contains a C-terminal amide group.

31. The method of any one of claims 21-30, wherein the first, second, and/or third peptide are each independently 8-50 amino acids in length.

32. The method of any one of claims 1 to 31, wherein the method further comprises comparing the T cell response measured in the first sample with the T cell response measured in the second sample to identify or aid in identifying the subject as having Celiac disease or as being in need of other testing if the T cell response measured in the first sample is elevated compared to the T cell response measured in the second sample, or to identify or aid in identifying the subject as not having or unlikely of having Celiac disease or as not having or unlikely of being in need of other testing if the T cell response measured in the first sample is substantially the same or decreased compared to the T cell response measured in the second sample.

33. The method of claim 32, wherein the method further comprises: treating the subject with a therapy if the subject is identified as having Celiac disease or recommending or providing information about a therapy to the subject.

34. The method of claim 33, wherein the therapy is a gluten-free diet.

35. The method of any one of claims 1 to 34, wherein the method further comprises performing another test on the subject prior to or after the steps of the method, preferably, in some embodiments, performing a serology and/or genotyping assay.

36. The method of claim 35, wherein the performing a serology and/or genotyping assay occurs prior to all of the steps recited in the method.

37. The method of claim 35, wherein the performing a serology and/or genotyping assay occurs after all of the steps recited in the method.

38. The method of any one of claims 1 to 37, wherein the subject is suspected of having Celiac disease.

39. The method of any one of claims 1 to 38, wherein the subject is HLA-DQ2.5 positive.

40. The method of any one of claims 1 to 39, wherein the first sample is obtained from the subject at least one day after administration of the first composition and the second sample is obtained from the subject at least one day after administration of the placebo.

41. The method of claim 40, wherein the first sample is obtained from the subject six days after administration of the first composition and the second sample is obtained from the subject six days after administration of the placebo.

42. A kit, comprising a composition comprising a gluten peptide and a placebo.

43. The kit of claim 42, wherein the composition and the placebo are foodstuffs.

44. The kit of claim 42 or 43, wherein the composition comprises at least one of a wheat gluten, a barley hordein, and a rye secalin.

45. The kit of claim 44, wherein the composition comprises at least two of a wheat gluten, a barley hordein, and a rye secalin.

46. The kit of claim 45, wherein the composition comprises a wheat gluten, a barley hordein, and a rye secalin.

47. The kit of any one of claims 42-46, wherein the composition comprises a container, such as a vial or tube, for whole blood.

48. The kit of claim 47, wherein one or more gluten peptides are dried on the wall of the container for whole blood.

49. The kit of claim 47, wherein one or more gluten peptides are in solution or lyophilized in a separate container.

50. The kit of claim 48 or 49, wherein the one or more gluten peptides are as defined in any of claim 31.

51. The kit of any one of claims 42-50, further comprising an anticoagulant.

52. The kit of any one of claims 47-51, wherein the container for whole blood and/or other container are present in duplicate or triplicate.

53. The kit of any one of claims 42-52, wherein the kit further comprises a negative control container, such as a vial or tube.

54. The kit of any one of claims 42-53, wherein the kit further comprises a positive control container, such as a vial or tube.

55. The kit of claim 53 or 54, wherein the negative and/or positive control container(s) are present in duplicate or triplicate.
